SEQUENCE LISTING (4 sequences)

SEQ ID NO: 1 - DNA, 20 bp, Artificial Sequence
  Synthetic
ggttaaacccagcttgacca

SEQ ID NO: 2 - DNA, 20 bp, Artificial Sequence
  Synthetic
ggaggcagagacagttggag

SEQ ID NO: 3 - DNA, 23 bp, Artificial Sequence
  Synthetic
gaagcagaagcttaggaagatgg

SEQ ID NO: 4 - DNA, 20 bp, Artificial Sequence
  Synthetic
ttggccccttaccataactg

(19) United States
(12) Patent Application Publication
(10) Pub. No.: US 2017/0056422 A1
(43) Pub. Date: Mar. 2, 2017

(54) PLASMODIUM LIVER-STAGE INHIBITORS AND RELATED METHODS

(71) Applicant: Seattle Biomedical Research Institute, Seattle, WA (US)

(72) Inventors: Alexis Kaushansky, Seattle, WA (US); Stefan H.I. Kappe, Seattle, WA (US); Alyse N. Douglass, Seattle, WA (US); Heather S. Kain, Seattle, WA (US)

(73) Assignee: Seattle Biomedical Research Institute, Seattle, WA (US)

(21) Appl. No.: 15/309,127

(22) PCT Filed: May 7, 2015
(86) PCT No.: PCT/US2015/029787

§ 371 Date: Nov. 4, 2016

Related U.S. Application Data
(60) Provisional application No. 61/989,838, filed on May 7, 2014.

(51) Int. Cl. A61K 31/635; A61K 31/404; A61K 31/4439; A61K 31/496

(52) U.S. Cl. CPC A61K 31/635; A61K 31/496; A61K 31/404; A61K 31/4439

(57) ABSTRACT

The present disclosure provides for methods of addressing the devastating effects of malaria infection by mosquito-borne Plasmodium parasites. The methods include the administration of an effective amount of at least one pro-apoptotic agent and the administration of an effective amount of at least one p53 activator. The at least one pro-apoptotic agent can be administered concurrently with, prior to, or subsequent to the at least one p53 activator. The at least one pro-apoptotic agent and/or at least one p53 activator can be administered concurrently with, prior to, or subsequent to exposure of a hepatocyte (in vivo or in vitro) by a Plasmodium parasite. In some embodiments, the administration of the at least one pro-apoptotic agent combined with the administration of the at least one p53 activator results in clearance of hypnozoite stage of P. vivax or P. ovale, and thus prevents relapse of symptoms and disease from the infection of these parasites.

### CROSS-REFERENCE TO RELATED APPLICATION

This application claims the benefit of Provisional Application No. 61/989,838, filed May 7, 2014, with is expressly incorporated herein by reference in its entirety.

### STATEMENT OF GOVERNMENT LICENSE RIGHTS

This invention was made with government support under F32AI091129 and 1R01GM101183-01A1 awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 53817_Sequence_Final_2015-05-06.txt. The text file is 2 KB; was created on May 7, 2015; and is being submitted via EFS-Web with the filing of the specification.

### BACKGROUND

Parasites of the genus Plasmodium, the causative agents of malaria, are transmitted to a host through the saliva of an infected Anopheles mosquito. After transmission, Plasmodium parasites, in the sporozoite stage, travel quickly through the blood stream to the liver. Sporozoites that infect hepatocytes can grow, replicate, and spawn tens of thousands of haploid daughters, called merozoites, into the blood stream. The vast amplification at this life cycle stage results, in part, due to their ability to evade detection by the host. The daughter merozoites that infect red blood cells cause the symptomatic infection of the host. Some species of the parasite, particularly Plasmodium vivax and Plasmodium ovale, can lie dormant in the host liver for months or years as hypnozoites, causing persistent, reoccurring symptomatic infection.

The asymptomatic hepatic stage of Plasmodium infection is an attractive malaria prophylactic intervention target, since during this stage few parasites are present and clinical symptoms are absent. Decades ago, it was found that irradiated Plasmodium sporozoites confer sterile, protective immunity in both rodents and humans. This was surprising, as a natural infection with malaria does not induce protective immunity in endemic areas of the world. Unfortunately, complications producing consistent batches of irradiated sporozoites and variable immunogenicity of irradiated sporozoite preparations have made this an impractical approach to the development of a vaccine. More recently, it has been demonstrated that sterile protective immunity is achieved after vaccination with genetically attenuated parasites (GAPs) in the rodent malaria models.

The molecular responses in the hepatocyte that occur during Plasmodium-parasite infection and the possible impact of these responses on parasite survival remain largely unknown. One attribute that has been partially uncovered is the ability of the malaria parasite to render its host hepatocyte resistant to some forms of apoptosis during liver-stage development (van de Sand, C., et al., “The Liver-Stage of Plasmodium berghei Inhibits Host Cell Apoptosis,” Mol Microbiol 58:731-742 (2005); Leiriao, P., et al., “HGF/MET Signaling Protects Plasmodium-Infected Host Cells From Apoptosis,” Cell Microbiol 7:603-609 (2005)). Elucidating the host responses triggered by Plasmodium parasite infection has been difficult due to differences among rodent and human Plasmodium species (Kaushansky, A. and Kappe, S. H., “The Crucial Role of Hepatocyte Growth Factor Receptor During Liver-Stage Infection Is Not Conserved Among Plasmodium Species,” Nat Med 17:1180-1181 (2011); Silvie, O., et al., “Hepatocyte CD81 is Required for Plasmodium falciparum and Plasmodium yoelii Sporozoite Infectivity,” Nat Med 9:93-96 (2003); Sattabongkot, J., et al., “Establishment of a Human Hepatocyte Line That Supports in vitro Development of the Exo-Erythrocytic Stages of the Malaria Parasites Plasmodium falciparum and P. vivax,” Am J. Trop Med Hyg 74(5):708-715 (2006)).

The inventors have previously shown that parasites actively suppress the tumor suppressor p53 (Kaushansky, A., et al., “Suppression of Host p53 is Critical for Plasmodium Liver-Stage Infection,” Cell Reports 3:630-637 (2013), incorporated herein by reference in its entirety), which plays a well-known role in promoting apoptosis and arresting cell cycle progression. Furthermore, the inventors have also shown that malaria parasites modulate members of the mitochondrial apoptotic cascade by increasing levels of the pro-survival factor B-cell lymphoma 2 (Bcl-2) and suppressing levels of the pro-apoptotic factor Bad (Kaushansky, A., et al., “Malaria Parasite Liver Stages Render Host Hepatocytes Susceptible to Mitochondria-Initiated Apoptosis,” Cell Death & Disease 4:e762 (2013), incorporated herein by reference in its entirety). Bcl2-inhibitors and p53 activators dramatically reduce liver stage burden both in vitro and in vivo. However, neither increasing p53 nor suppressing Bcl-2 family members alone appears to be sufficient to fully eliminate the parasite at the liver stage and completely prevent development of blood stage malaria.

Even in view of the advances in the art, there remains a need for drugs and therapy strategies that eliminate the parasite in the liver. Moreover, drug resistance to anti-malarial drugs, including primaquine and other drugs that target the blood-stage disease, presents a major obstacle towards prophylaxis, treatment, and eradication of malaria, which indicate a further need in the art for drugs that eliminate or minimize the ability of the parasite to develop resistance to treatment. Finally, there remains a need for drugs and therapeutic strategies that prevent relapse of disease by preventing the development of hypnozoite stages, which have been heretofore recalcitrant to treatment.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method for inhibiting growth or development of a liver-stage Plasmodium parasite in a hepatocyte. The method comprises administering to the hepatocyte an effective amount of at least one pro-apoptotic agent and an effective amount of at least one p53 activator.

In one embodiment, the at least one pro-apoptotic agent promotes the mitochondrial apoptotic cascade. In one embodiment, the at least one pro-apoptotic agent inhibits expression or function of a Bcl-2 family protein. In one embodiment, the at least one pro-apoptotic agent inhibits expression or function of two or more Bcl-2 family proteins. In one embodiment, the at least one pro-apoptotic agent inhibits functional binding of the BH-3 domain of the Bcl-2 family protein. In one embodiment, the Bcl-2 family protein is Bcl-xL or a homolog thereof. In one embodiment, the at least one pro-apoptotic agent inhibits the expression of the Bcl-2 family protein and is a histone deacetylase inhibitor, a retinoid, a cyclin-dependent kinase inhibitor, or any analog thereof, or an antisense nucleic acid molecule targeting a gene encoding the Bcl-2 family protein. In one embodiment, the at least one pro-apoptotic agent is gossypol, ABT-737, ABT-263, an indole bipyrrole such as GX15-070, HA14-1, antimycin, obatoclax, isoxazolidine, benzoyl urea, AT-101, TW-37, or any functional derivative or analog thereof.

In one embodiment, the at least one p53 activator increases the stability, expression, or activity of p53. In one embodiment, the at least one p53 activator is or includes 9AA, a canbinol, an HLI98 series molecule, a JJ78:1/12 series molecule, a tenovin, CDB3, KCG165, an aminothiosol, or RITA, as recited in Table 1. In one embodiment, the at least one p53 activator inhibits or reduces the interaction of p53 with Mdm2 or MdmX. In one embodiment, the at least one p53 activator is or includes a benzodiazepine, a benzodiazepinedone, a chromenotrizolopyrimindine, a dehydroaltenusin, an imidazole-indole, a spiro-oxindole, an imidazoline, an oxindole, a spiroindolinone, an isoquinolines, a bisaryl sulfonamide, a substituted piperidine, a diphenyl-dihydro-imidazopyridinone, an imidazothiazole, a deazaflavin, an isoindolin-1-one, boronic acid, a pyrrolidin-2-one, SJ172550, or a tryptamine, as recited in Table 1. In one embodiment, the at least one p53 activator is or includes Nutlin-3 or Serdemetan.

In one embodiment, the effective amount of the at least one pro-apoptotic agent and/or the effective amount of the at least one p53 activator is administered prior to exposure of the hepatocyte to a Plasmodium parasite. In one embodiment, the effective amount of the at least one pro-apoptotic agent and/or the effective amount of the at least one p53 activator is administered concurrently with or subsequent to exposure of the hepatocyte to a Plasmodium parasite. In one embodiment, the effective amount of the at least one pro-apoptotic agent is administered concurrently with the effective amount of the at least one p53 activator. In one embodiment, the therapeutically effective amount of the at least one pro-apoptotic agent is administered prior to or subsequent to the administration of the effective amount of the at least one p53 activator.

In one embodiment, the liver-stage Plasmodium parasite is P. falciparum, P. vivax, P. ovale, P. malariae, P. knowlesi, P. yoelii, P. berghei, P. chabaudi, P. vinckei, or P. cynomolgi. In one embodiment, the liver-stage Plasmodium parasite is a hypnozoite of P. vivax or P. ovale. In one embodiment, liver-stage Plasmodium parasite is a drug-resistant Plasmodium parasite.

In one embodiment, the hepatocyte is cultured in vitro and the effective amounts of the at least one pro-apoptotic agent and the at least one p53 activator are administered to the culture. In another embodiment, the hepatocyte is in vivo in a vertebrate subject and the effective amounts of the at least one pro-apoptotic agent and the at least one p53 activator are administered to the vertebrate subject. In a further embodiment, the vertebrate is infected with a Plasmodium parasite or is susceptible to infection with a Plasmodium parasite. In a further embodiment, the vertebrate subject is a human subject.

In one embodiment, inhibiting growth or development of a liver-stage Plasmodium parasite results in the elimination of the liver-stage Plasmodium parasite from the hepatocyte.

In another aspect, the disclosure provides a method of inhibiting growth or development of a liver-stage Plasmodium parasite in a hepatocyte of a vertebrate subject. The method comprises administering to the subject an effective amount of at least one pro-apoptotic agent and an effective amount of at least one p53 activator.

In another aspect, the disclosure provides a method of preventing infection of a hepatocyte in a vertebrate subject by a liver-stage Plasmodium parasite. The method comprises administering to the subject an effective amount of at least one pro-apoptotic agent and an effective amount of at least one p53 activator.

In another aspect, the disclosure provides a method of preventing or reducing production of blood-stage Plasmodium parasite by a liver-stage Plasmodium parasite in a hepatocyte of a vertebrate subject. The method comprises administering to the subject an effective amount of at least one pro-apoptotic agent and an effective amount of at least one p53 activator.

In another aspect, the disclosure provides a method of generating protective immunity against a Plasmodium parasite in a vertebrate subject. The method comprises administering to the subject an effective amount of at least one pro-apoptotic agent and an effective amount of at least one p53 activator.

Any of the above aspects include embodiments where the effective amounts of the at least one pro-apoptotic agent and/or the at least one p53 activator are administered to the subject prior to, concurrently with, or subsequent to infection of a hepatocyte of the subject with the Plasmodium parasite. Further, in some embodiments of any of the above aspects, the vertebrate subject is a human. In some embodiments of the above aspects, the liver-stage Plasmodium parasite is a hypnozoite of P. vivax or P. ovale. In further embodiments, the administration of the effective amounts of the at least one pro-apoptotic agent and the at least one p53 activator results in elimination of the hypnozoites from the subject.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1E are structural illustrations of small molecules targeting p53 or Bcl-2 family proteins. FIG. 1A illustrates the structure of Nutlin-3 and FIG. 1B illustrates the structure of Serdemetan, which increase p53 levels by preventing binding with MDM-2. FIG. 1C illustrates the structure of Obatoclax-mesylate, which inhibits the pro-survival BH3 proteins Bcl-2, Bcl-xL, and Mcl-1. FIG. 1D illustrates the structure of ABT-737, which inhibits Bcl-2, Bcl-w, and Bcl-xL. FIG. 1E illustrates the structure of ABT-199, which inhibits Bcl-2 only.

FIGS. 2A-2E illustrate that Bcl-2 family inhibition and p53 clear LS parasites by independent mechanisms. FIGS. 2A and 2B graphically illustrate the number of LS parasites after treatment with p53 agonist or Bcl2-inhibitors. Hepa 1-6 cells were infected with P. yoelii and treated with Nutlin-3 (20 μM), ABT-737 (100 nM), Obatoclax (100 nM) with or without the pan-caspase inhibitor qVD-OPh (20 μM). After 24 (FIG. 2A) or 48 (FIG. 2B) hours, cultures were fixed and LS parasites were identified by UIS4 and HSP70 expression and quantified microscopically. FIGS. 2C and 2D illustrate immunoblots showing p53 expression after treatment with p53 agonist or Bcl2-inhibitors. Hepa 1-6 cells were treated with Nutlin-3 (20 μM), ABT-737 (100 nM), or Obatoclax (100 nM) for 24 (FIG. 2C) or 48 (FIG. 2D) hours. Protein levels of p53 were determined by immunoblot. FIG. 2E schematically illustrates a model for elimination of LS parasites by Obatoclax (“apoptotic death”) and Nutlin-3 (“nonapoptotic death”). Generally, NS=(not significant), *=(P≦ 0.05), **=(P≦ 0.01).

FIG. 3 graphically illustrates the percentage of permeabilized cells after treatment with Obatoclax and Nutlin-3. The results demonstrate that Obatoclax and Nutlin-3 do not cause cell death in uninfected hepatocytes in vitro. Hepa 1-6 cells were treated with DMSO (0.1%), Staurosporine (2 μM), Obatoclax (100 nM), or Nutlin-3 (20 μM) for 48 hours and analyzed for cell death with a permeability dye by flow cytometry.

FIGS. 4A and 4B graphically illustrate the number of LS parasites per well after treatment with Bcl-2 family inhibitors. The data demonstrate that Bcl-2 inhibition alone is not able to eliminate P. yoelii LS in vitro. Both ABT-737 and Obatoclax inhibit more than one Bcl-2 family member (Bcl-2, Bcl-xL and Bcl-w; Bcl-2, Bcl-xL and Mcl-1, respectively), although both compounds inhibit the BH3 domain of Bcl-2 to the greatest degree. To determine if inhibition of Bcl-2 alone (ABT-199) was sufficient to eliminate P. yoelii LS parasites, Hepa 1-6 cells were infected for 24 (FIG. 4A) or 48 (FIG. 4B) hours and treated with DMSO (0.1%), ABT-199 (100 nM), ABT-737 (100 nM), or Obatoclax (100 nM) for the full length of infection. Parasites were identified by UIS4 and Hsp70 expression and quantified microscopically.

FIGS. 5A and 5B graphically illustrate the days to patency in vivo observed for Plasmodium when initial infection occurs after administration of a Bcl-2 family inhibitor, a p53 agonist, and a combination thereof. The data demonstrate that the combination of Nutlin-3 and Obatoclax dramatically delays, or in some cases completely prevents, the onset of disease in vivo. BALB/cJ mice were treated with vehicle, Nutlin-3 (200 mg/kg/day), Obatoclax (5 mg/kg/day), or a combination of Nutlin-3 and Obatoclax beginning 24 hours prior to infection and then once daily for 3 days total. Mice were infected intravenously with 1,000 (FIG. 5A) or 100 (FIG. 5B) P. yoelii sporozoites. Blood stage patency was monitored by Giemsa-stained thin blood smear beginning 3 days after infection and continuing through 2 weeks post-infection.

FIG. 6 graphically illustrates the days to patency for Plasmodium parasites in mice treated with different p53 genetic backgrounds. The data demonstrate that Nutlin-3 specifically increases p53 levels to clear P. yoelii LS in vivo. Wild-type (cre-Alb) and liver-specific p53 knock-out (p53-flox/cre-Alb) mice were treated with vehicle or Nutlin-3 (200 mg/kg) twice daily for four days. On the second day of treatment, all mice were intravenously infected with 10³ P. yoelii spz and checked for patency once daily by Giemsa-stained thin blood smear starting three days post-infection and continuing until all mice became patent.

FIGS. 7A-7D illustrate the finding that Serdemetan treatment decreases P. yoelii LS burden in Hepa 1-6 cells in a dose-dependent manner. FIG. 7A graphically illustrates the number of LS parasites after treatment of various doses of Serdemetan. Hepa 1-6 cells were infected with P. yoelii sporozoites in chamber slides and treated with the indicated concentration of Serdemetan for 48 hours. Parasites were quantified microscopically by staining with UIS4 and HSP70. FIG. 7B illustrates an immunoblot showing p53 expression after treatment of Serdemetan. Hepa 1-6 cells were treated with Serdemetan (10 μM) for 48 hours and analyzed for p53 expression by immunoblot. FIG. 7C graphically illustrates the number of LS parasites after treatment with Serdemetan with or without caspase inhibitor qVD-OPh. Hepa 1-6 cells were infected with P. yoelii and treated with Serdemetan (10 μM) with or without the total caspase inhibitor qVD-OPh (20 μM) for 24 hours. FIG. 7D graphically illustrates the number of LS parasites after treatment with Serdemetan at different times relative to initial infection. Hepa 1-6 cells were infected with P. yoelii parasites and LS burden was quantified at 24 hours post-infection by microscopy as described for FIG. 7C. Parasites were either treated with Serdemetan 24 hours before infection (−24 hours), 24 hours after infection (+24 hours), or both (−24 hours to +24 hours). Generally, NS=(not significant), *=(P≦ 0.05), **=(P≦ 0.01) when compared to non-treated controls.

FIGS. 8A and 8B graphically illustrate the complete elimination of LS burden in vitro and in vivo by a combination treatment of Serdemetan and Obatoclax. FIG. 8A graphically illustrates the number of LS parasites with Serdemetan, Obatoclax, or a combination of Serdemetan and Obatoclax. Hepa 1-6 cells were infected with P. yoelii sporozoites in chamber slides and treated with Serdemetan (10 μM), Obatoclax (100 nM), Serdemetan and Obatoclax, or media only (NT) for 48 hours. LS parasites were identified by UIS4 and HSP70 expression using fluorescence microscopy and quantified. NS (not significant), *(P≦ 0.05), **(P≦ 0.01) when compared to non-treated controls. FIG. 8B graphically illustrates the days to patency after Plasmodium infection in mice in the context of different treatment regiments. BALB/cJ mice receiving the “standard treatment” were treated 24 hours prior to infection, and every day for five days (including the day of infection) with vehicle only, Serdemetan (20 mg/kg/day) only, Serdemetan (20 mg/kg/day) and Obatoclax (5 mg/kg/day), or double Serdemetan (40 mg/kg/day) and Obatoclax (5 mg/kg/day). There was an additional condition where mice were treated with Serdemetan (20 mg/kg/day) and Obatoclax (5 mg/kg/day) according to the standard treatment, described above, plus receiving additional treatment every day for an additional five (“extended treatment”). Patency was monitored for all conditions by thin blood smear and Giemsa stain.

FIG. 9 graphically illustrates that the FRG HuHep mice can model the effectiveness of anti-malarial treatments against P. falciparum. FRG HuHep mice were treated with either vehicle or Atovaquone (10 mg/mL) by oral gavage once daily for three days. On the second day of treatment, mice were infected (i.v.) with 10⁶ P. falciparum GFP-Luc transgenic parasites and infection was monitored by IVIS days 4, 5, and 6 post-infection.

FIGS. 10A and 10B illustrate that P. falciparum is cleared from the liver of humanized mice treated with Serdemetan and Obatoclax. FRG HuHep mice were treated with either vehicle control or both 5 mg/kg of Obatoclax and 20 mg/kg Serdemetan by oral gavage once daily for 8 days. On the second day of treatment, mice were injected i.v. with 10⁶ P. falciparum GFP-luc transgenic parasites. Parasite load was assessed by IVIS. FIG. 10A is a pictoral depiction of representative mice with superimposed images of detected light output indicating parasite burden (see mouse on the left). FIG. 10B graphically represents the light output observed for mice receiving vehicle or the combination treatment. Mice with undetected parasite burdens are depicted with an open circle.

FIGS. 11A-11D illustrate that Obatoclax and Serdemetan do not cause non-specific cell death or toxicity either in vivo or in vitro. FIG. 11A graphically illustrates the percent of permeabilized (dead) cells treated with various agents. Hepa 1-6 cells were treated with DMSO (0.1%), Staurosporine (2 μM), Obatoclax, (100 nM), Serdemetan (10 μM), or Obatoclax and Serdemetan for 48 hours and analyzed for cell death with a permeability dye by flow cytometry. FIG. 11B graphically illustrates the ALT levels from mice treated with vehicle, Obatoclax (5 mg/kg), Serdemetan (20 mg/kg), or Obatoclax and Serdemetan by oral gavage once daily for 5 days were assessed 2 weeks following treatment. FIGS. 11C and 11D are photographs that depict liver damage from FRG HuHep mice treated with either vehicle or Obatoclax (5 mg/kg) and Serdemetan (20 mg/kg) in combination for 8 days was assessed by H&E staining and microscopic analysis.

FIG. 12 graphically illustrates the liver stage schizonts and hypnozoites of Plasmodium vivax present in liver cells from FRG HuHep mice with or without combined treatment with Obatoclax and 20 mg/kg Serdemetan.

### DETAILED DESCRIPTION

The present application discloses the inventors' discovery that Bcl2-inhibitors and p53 agonists can affect parasite loads by acting through different pathways. When combined, these therapeutic agents were surprisingly found to greatly reduce or completely eliminate the liver stage (LS) parasites. This is likely the result of a synergistic effect of the Bcl2-inhibitors and p53 agonists as they target both pathways during liver-stage infection, thereby providing a novel, host-based, prophylactic intervention strategy for malaria. Furthermore, this approach also had the unexpected and unprecedented effect of completely eliminating hypnozoites of Plasmodium vivax, which provides a novel and powerful tool for the long-term prevention of disease relapse. As described in more detail below, the use of at least one agent that inhibits Bcl-2 family members together with at least one agent that increases levels of functional p53 results in complete elimination of liver stage Plasmodium parasites in a humanized mouse model. By targeting two independent host pathways, one regulated by Bcl-2 family proteins and the other by p53, blood stage disease can be entirely prevented. Additionally, addressing Plasmodium infections through host-based prophylactic (HBP) strategies (e.g., elimination of the parasite in the hepatocyte through modulation of host factors rather than directly attacking the parasite) likely provides the additional benefit of reducing the opportunity for the parasite to develop drug resistance to the treatment. Moreover, it was discovered that this combined HBP strategy also clears liver cells of P. vivax hypnozoites, which have been notoriously recalcitrant to extant malarial therapies that target the liver stage parasites directly. Accordingly, the combination of Bcl2-family inhibitors and p53 agonists are a powerful new tool to eliminate the risk of disease relapse.

Traditional approaches to malaria drug development, including drug development against liver stages of the parasite, focus on discovery of drugs that are active against the parasite and have little or no activity on the host cells. Meister, S., et al., “Imaging of Plasmodium Liver Stages to Drive Next-Generation Antimalarial Drug Discovery,” Science 334(6061):1372-1377 (2011). The goal is to reduce the parasite burden with minimal damage to the hepatocyte. Here, the inventors have taken a novel approach to malaria drug discovery by preferentially targeting the host factors required for parasite survival. Remarkably, by using both Bcl-2 family inhibitors and p53 agonists in combination, one of the drugs can act as a sensitization agent to cause the parasite to rely more heavily on the other host pathway, and then the second drug can eliminate the “sensitized” parasite. This approach has been described to selectively promote apoptosis in cancer cells, namely by using drugs that target the Bcl-2 family pathways as sensitization agents in combination with traditional chemotherapy. Letai, A. G., “Diagnosing and Exploiting Cancer's Addition to Blocks in Apoptosis,” Nature Reviews: Cancer 8:121-132 (2008), incorporated herein by reference in its entirety. However, exploiting the parasite's addiction to certain host pathways by selectively targeting such pathways with drug treatment is an innovative approach to malaria prophylaxis and treatment that has not been explored in the malaria field.

Several apoptotic pathways have been uncovered, and one of the most important involves the Bcl-2 family of proteins, which are key regulators of the mitochondrial (also called “intrinsic”) pathway of apoptosis (see, e.g., Danial & Korsmeyer, “Cell Death: Critical Control Points,” Cell 116:205-219 (2004); Kang & Reynolds, “Bcl-2 Inhibitors: Targeting Mitochondrial Apoptotic Pathways in Cancer Therapy,” Clin. Cancer Res. 15(4):1126-1132 (2009), incorporated herein by reference in their entireties). The structural homology domains BH1, BH2, BH3 and BH4 were first described for Bcl-2 (hence the use of the term “Bcl-2 family”), but the BH3 domain is the hallmark of this family of proteins. In the broadest sense, the Bcl-2 family of proteins can be further classified into three subfamilies depending on how many of the homology domains each protein contains and on its biological activity (i.e., whether it has pro- or anti-apoptotic function).

The first subgroup contains proteins having all four homology domains, i.e., BH1, BH2, BH3 and BH4. Proteins such as, for example, Bcl-2, Bcl-w, Bcl-xL, Mcl-1, Bcl-b, and Bfl-1/A1 are members of this first subgroup. Because this subgroup contains the specific Bcl-2 protein, this subgroup is also often referred to specifically as the Bcl-2 family, as distinct from the other subgroups described below. The general effect of this first subgroup of proteins is anti-apoptotic, that is, to preserve a cell from starting a cell death process. This effect is in part due to their ability to inhibit the apoptotic activity of the second subgroup, described below. Proteins belonging to the second subgroup contain the three homology domains BH1, BH2 and BH3, and have a pro-apoptotic effect. The two main representative proteins of this second subgroup are Bax and Bak, and proteins working in a similar fashion, such as Bok. Finally, the third subgroup is composed of proteins containing only the BH3 domain and members of this subgroup are usually referred to as “BH3-only proteins.” Their biological effect on the cell is pro-apoptotic, by interfering with the inhibiting effect of the first subgroup. Bim, Bid, Bad, Bik, Noxa, Hrk, Bmf, and Puma are examples of this third subfamily of proteins (see, e.g., Azmi et al., “Emerging Bcl-2 Inhibitors for the Treatment of Cancer,” Expert Opin Emerg Drugs 16(1):59-70 (2011), incorporated herein by reference in its entirety).

Unless indicated otherwise, the term “Bcl-2 family” as used herein below refers to the anti-apoptotic subgroup of the general Bcl-2 family, wherein the family members comprise all four homology domains (BH1, BH2, BH3, and BH4). Moreover, for clarity it is noted that the term “Bcl-2 family” is not limited specifically to the Bcl-2 protein and its associated pathway, but rather refers to the entire group of related proteins in this anti-apoptotic subgroup (i.e., first subgroup as described above) and their associated pathways.

As described in more detail below, p53 activators appear to eliminate parasites via an apoptosis-independent pathway. The abundance and activity of p53 is tightly regulated via a number of mechanisms: transcription, translation, protein stability, post-translational modification, degradation and intracellular localization (see, e.g., Brady, C. A. and Attardi, L. D., “p53 at a Glance,” J Cell Sci 123:2527-2532 (2010); Mandinova, A. and Lee, S., “The p53 Pathway as a Target in Cancer Therapeutics: Obstacles and Promise,” Sci Trans Med, 3:64:1-7 (2011); Popowicz, G., et al., “The Structure-Based Design of Mdm2/Mdmx Inhibitors Gets Serious,” Angew Chem Int Ed 50:2680-2688 (2011), each incorporated herein by reference in its entirety). The activation of p53 by phosphorylation turns p53 into a transcriptional regulator that alters the expression of specific genes. The DNA binding ability of p53 is enhanced by acetylation. Thus, deacetylation of p53 by the SIRT family decreases p53 activity (Langley, E., et al., “Human SIR2 Deacetylates p53 and Antagonizes PML/p53—Induced Cellular Senescence,” EMBO J 21(10):2383-2396 (2002), incorporated herein by reference in its entirety). The activity of p53 is also negatively regulated by Mdm2 (also known as Hdm2) and Mdmx. In addition, the levels of p53 are negatively regulated, for example, by Mdm2, Pirh2 and Cop1.

Without being bound to any particular theory, the investigations described herein indicate that the liver stage of Plasmodium parasites are rely on (i.e., are “addicted to”) the Bcl-2 family pathway for its survival and development within the host hepatocyte, akin to various cancer cells becoming reliant on the activity of specific anti-apoptotic pathways. Accordingly, the present disclosure provides for methods that address Plasmodium infection by targeting this pathway to sensitize the parasite with a pro-apoptotic agent, which renders the parasite susceptible to further treatment with, for example, a p53 activator.

In accordance with the above, the present disclosure provides methods of preventing infection of hepatocytes by Plasmodium parasites, methods for inhibiting the growth or development of liver stage Plasmodium parasites, methods for treating liver-stage infection, methods for preventing malaria, and methods for inducing protective immunity in hepatocytes. Additionally, this invention provides methods of preventing infection of hepatocytes by drug-resistant Plasmodium parasites, methods for inhibiting the growth or development of liver stage drug-resistant Plasmodium parasites, methods for treating liver-stage infection resulting from drug-resistant Plasmodium parasites, and methods for preventing drug-resistant malaria. These methods comprise the step of administering to a vertebrate subject in need thereof an effective amount of at least one pro-apoptotic agent combined with (or coordinated with) administering to the vertebrate subject in need at least one p53 activator, or the step of contacting a hepatocyte, prior to, concurrently with, or subsequent to infection with a Plasmodium parasite or a drug-resistant Plasmodium parasite, with an effective amount of at least one pro-apoptotic agent and at least one p53 activator.

As used herein, the terms “Plasmodium parasite” or “parasite” refer to any parasite that belongs to the genus Plasmodium. In some embodiments, the Plasmodium parasite can infect human hosts, such as, for example, P. falciparum, P. vivax, P. ovale, P. malariae, and P. knowlesi. In some embodiments of the invention, the Plasmodium parasite is P. falciparum. In some embodiments, the Plasmodium parasite is P. vivax or P. ovale. In other embodiments, the Plasmodium parasite can infect other vertebrate hosts, such as non-human primates and rodents. Examples of such Plasmodium parasites include P. yoelii, P. berghei, P. chabaudi, P. vinckei, and P. cynomolgi.

As used herein, the terms “drug-resistant Plasmodium parasite” or “drug resistant parasite” refer to a Plasmodium parasite that is resistant to at least one approved anti-malarial drug. In this context, the approved anti-malarial drugs are distinguished from the presently disclosed treatment strategy because, as described herein, the presently disclosed strategy is believed to be unlikely to result in resistance by the parasite because the strategy addresses the environment provided by the host hepatocyte and does not directly address the parasite itself. Accordingly, the presently disclosed strategy is useful to address infections by Plasmodium parasites that have developed resistance to traditional drugs. In some embodiments, the drug-resistant Plasmodium parasite is chloroquine-resistant. In other embodiments, the drug-resistant Plasmodium parasite is primaquine-resistant. In some embodiments, the drug-resistant Plasmodium parasite is artemisinin-resistant. In some embodiments, the drug-resistant Plasmodium parasite is doxycycline-resistant. In yet other embodiments, the drug-resistant Plasmodium parasite is atovaquone-resistant. In some embodiments, the drug-resistant Plasmodium parasite is mefloquine-resistant. In further embodiments, the drug-resistant Plasmodium parasite is resistant to more than one anti-malarial drug. In some embodiments, the drug-resistant parasite is resistant to atovaquone and proguanil hydrochloride.

The term “liver stage” or “LS” refers to the phase of the Plasmodium life cycle that occurs within hepatocytes, starting after the invasion of a hepatocyte by an infective sporozoite and ending with the release of merozoites. As used herein, liver stages include the schizonts as well as dormant LS (i.e., “hypnozoites”) of P. vivax, P. ovale, and P. malariae.

As used herein, the term “vertebrate subject” includes, but is not limited to, a mammalian host that is susceptible to infection by a Plasmodium parasite or a drug-resistant parasite. Such mammalian hosts include primates, such as a human subject, and rodents, such as a murine or rat subject. In certain specific embodiments, the vertebrate subject is a human subject. Those in need of treatment include those already with the disease, as well as those prone to have the disease, or those in whom the disease is to be prevented. Accordingly, in some embodiments the subjects to be treated are human subjects infected with one or more species of Plasmodium parasites or drug-resistant parasites and, in some embodiments, the subjects to be treated are human subjects at risk for being infected with one or more species of Plasmodium parasites or drug-resistant parasites.

As used herein, the term “p53 activator” refers to any agent that activates the p53 response to render hepatocytes more resistant to Plasmodium parasite or drug-resistant parasite infection, to inhibit the development of Plasmodium parasite or drug-resistant parasite liver-stages, and/or to promote apoptosis of Plasmodium-infected or drug-resistant Plasmodium-infected hepatocytes.

In some embodiments, the p53 activator increases the expression levels of p53, directly or indirectly. In some embodiments, the p53 activator increases or promotes the stability of expressed p53 directly or indirectly. In some embodiments, the “promoting the stability of expressed p53” includes preventing the degradation of p53 directly or indirectly. In some embodiments, the p53 activator increases the activity of p53 directly or indirectly. In some embodiments, the p53 activators used in the methods of the invention inhibit the interaction of p53 with Mdm2 or MdmX, directly or indirectly. For example, indirect inhibition of interaction of p53 with Mdm2 or MdmX can include downregulation of Mdm2 or MdmX.

In some embodiments of the invention, the p53 activator is a small drug-like molecule. In other embodiments, the p53 activator may be a peptide, antibody, microRNA, or macromolecule (see, e.g., Dömling A., “Small Molecular Weight Protein-Protein Interaction Antagonists—An Insurmountable Challenge?” Curr Opin Chem Biol 12:281-91 (2008) and U.S. 20100104662, each reference hereby incorporated by reference in its entirety).

Exemplary p53 activators suitable for use in the methods of the present invention are well-known and have been previously described and some of these have entered clinical trials for the treatment of cancer (see, e.g., in Mandinova, A. and Lee, S., “The p53 Pathway as a Target in Cancer Therapeutics: Obstacles and Promise,” Sci Trans Med 3:1-7 (2011); McCarthy, A., et al., “The Discovery of Nongenotoxic Activators of p53: Building on a High-Throughput Screen,” Seminars in Canc Bio 20:40-45 (2010); Popowicz, G., et al., “The Structure-Based Design of Mdm2/Mdmx Inhibitors Gets Serious,” Angew Chem Int Ed 50:2680-2688 (2011); Wang, Z. and Sun, Y., “Targeting p53 for Novel Anticancer Therapy,” Trans Onc 3:1-12 (2010); Weber, L., “Patented Inhibitors of p53-Mdm2 Interaction (2006-2008)” Expert Opin Ther Pat 20(2):179-191 (2010); US Patent Application Publication Nos. 20080280769, 20050227932, 20090143364, 20050288287, 20110112052, 20090312310, 20060211718, 20100048593, 20080261917, 20090227542, 20080171723, 20060211757, 20050137137, 20020132977, 20090030181, 20110224274, 20110251252, 20110183917, 20100075949, 20110021529, 20100168388, 20100168388, 20090306130; WO 2008106507; and U.S. Pat. Nos. 7,964,724, 7,759,383, 7,060,713, 7,553,833, 6,916,833; 7,495,007; 7,638,548, 7,576,082, 7,625,895, 7,083,983, each of which are hereby incorporated by reference). Non-limiting examples of p53 activators useful in the practice of the invention are listed in Table 1 below:

TABLE 1
list of p53 activator classes and relevant references therefore

| Classes of p53 activators | | References (each of which are incorporated herein |
|---|---|---|
| (examples) | Activity | by reference in their entireties) |
| 9AA | Increase of p53 levels | McCarthy, A., et al., “The Discovery of Nongenotoxic |
| | | Activators of p53: Building on a High-Throughput |
| | | Screen,” Seminars in Canc Bio, 20: 40-45 (2010) |
| Benzodiazepines, | Inhibition of p53- | Popowicz, G., et al., “The Structure-Based Design of |
| benzodiazepinedones | Mdm2 interaction | Mdm2/Mdmx Inhibitors Gets Serious,” Angew Chem |
| (TDP222669, | | Int Ed, 50: 2680-2688 (2011); |
| TDP521252, | | Wang, Z. and Sun, Y., “Targeting p53 for Novel |
| TDP665759) | | Anticancer Therapy,” Trans Onc, 3: 1-12 (2010); |
| | | Weber, L. “Patented Inhibitors of p53-Mdm2 |
| | | Interaction (2006-2008),” Expert Opin Ther Pat, |
| | | 20(2): 179-91 (2010); |
| | | U.S. 20050227932 |
| Cambinols | Inhibition of | McCarthy, A., et al., “The Discovery of Nongenotoxic |
| | SIRT1/T2 | Activators of p53: Building on a High-Throughput |
| | | Screen,” Seminars in Canc Bio, 20: 40-45 (2010) |
| CDB3 | Stabilization of p53 | Wang, Z. and Sun, Y., “Targeting p53 for Novel |
| | | Anticancer Therapy,” Trans Onc, 3: 1-12 (2010) |
| Chromenotrizolopyrimindines | Inhibition of p53- | Popowicz, G., et al., “The Structure-Based Design of |
| | Mdm2 interaction | Mdm2/Mdmx Inhibitors Gets Serious,” Angew Chem |
| | | Int Ed, 50: 2680-2688 (2011) |
| Dehydroaltenusin | Inhibition of p53- | McCarthy, A., et al., “The Discovery of Nongenotoxic |
| | Mdm2 interaction | Activators of p53: Building on a High-Throughput |
| | | Screen,” Seminars in Canc Bio, 20: 40-45 (2010) |
| HLI98 series | Inhibition of Mdm2 | Wang, Z. and Sun, Y., “Targeting p53 for Novel |
| | activity | Anticancer Therapy,” Trans Onc, 3: 1-12 (2010) |
| Imidazole-indoles | Inhibition of p53- | Popowicz, G., et al., “The Structure-Based Design of |
| (WW298, WK23) | Mdmx interaction | Mdm2/Mdmx Inhibitors Gets Serious,” Angew Chem |
| | | Int Ed 50: 2680-2688 (2011); Weber, L., “Patented |
| | | Inhibitors of p53-Mdm2 Interaction (2006-2008),” |
| | | Expert Opin. Ther. Pat, 20(2): 179-91 (2010) |
| JJ78:1/12 series | Inhibition of tubulin | McCarthy, A., et al., “The Discovery of Nongenotoxic |
| | polymerization | Activators of p53: Building on a High-Throughput |
| | | Screen,” Seminars in Canc Bio, 20: 40-45 (2010); |
| | | Wang, Z. and Sun, Y., “Targeting p53 for Novel |
| | | Anticancer Therapy,” Trans Onc, 3: 1-12 (2010) |
| KCG165 | Increase of p53 levels | McCarthy, A. et al., “The Discovery of Nongenotoxic |
| | | Activators of p53: Building on a High-Throughput |
| | | Screen,” Seminars in Canc Bio, 20: 40-45 (2010) |
| Spiro-oxindoles | Inhibition of p53- | Mandinova, A. and Lee, S., “The p53 Pathway as a |
| (MI-63, MI-219, MI- | Mmd2 interaction | Target in Cancer Therapeutics: Obstacles and |
| 43, MI-319) | | Promise,” Sci Trans Med, 3: 64, 1-7 (2011); |
| | | Popowicz, G., et al. “The Structure-Based Design of |
| | | Mdm2/Mdmx Inhibitors Gets Serious,” Angew Chem |
| | | Int Ed, 50: 2680-2688 (2011); |
| | | Wang, Z. and Sun, Y., “Targeting p53 for Novel |
| | | Anticancer Therapy,” Trans Onc, 3: 1-12 (2010); |
| | | U.S. 2011/0112052; |
| | | U.S. Pat. No. 7,759,383 |
| Imidazolines | Inhibition of p53- | McCarthy, A., et al., “The Discovery of Nongenotoxic |
| (Nutlin-3, RG7112) | Mdm2 interaction | Activators of p53: Building on a High-Throughput |
| | | Screen,” Seminars in Canc Bio, 20: 40-45 (2010); |
| | | Mandinova, A. and Lee, S., “The p53 Pathway as a |
| | | Target in Cancer Therapeutics: Obstacles and |
| | | Promise,” Sci Trans Med, 3: 1-7 (2011); |
| | | Popowicz, G., et al. “The Structure-Based Design of |
| | | Mdm2/Mdmx Inhibitors Gets Serious,” Angew Chem |
| | | Int Ed, 50: 2680-2688 (2011); |
| | | Wang, Z. and Sun, Y., “Targeting p53 for Novel |
| | | Anticancer Therapy,” Trans Onc, 3: 1-12 (2010); |
| | | Weber, L. “Patented Inhibitors of p53-Mdm2 |
| | | Interaction (2006-2008),” Expert Opin Ther Pat, |
| | | 20(2): 179-91 (2010); |
| | | U.S. 2009/0143364, U.S. 2005/0288287; |
| | | U.S. Pat. No. 7,964,724 |
| RITA | Stabilization of p53 | Mandinova, A. and Lee, S., “The p53 Pathway as a |
| | | Target in Cancer Therapeutics: Obstacles and |
| | | Promise,” Sci Trans Med, 3: 1-7 (2011); |
| | | McCarthy, A. et al., “The Discovery of Nongenotoxic |
| | | Activators of p53: Building on a High-Throughput |
| | | Screen,” Seminars in Canc Bio, 20: 40-45 (2010); |
| | | Wang, Z. and Sun, Y., “Targeting p53 for Novel |
| | | Anticancer Therapy,” Trans Onc, 3: 1-12 (2010) |
| SJ172550 | Mdmx inhibitor | Mandinova, A. and Lee, S., “The p53 Pathway as a |
| | | Target in Cancer Therapeutics: Obstacles and |
| | | Promise,” Sci Trans Med, 3: 1-7 (2011) |
| Tenovins | Inhibition of | Mandinova, A. and Lee, S., “The p53 Pathway as a |
| | SIRT1/T2 | Target in Cancer Therapeutics: Obstacles and |
| | | Promise,” Sci Trans Med, 3: 1-7 (2011); |
| | | McCarthy, A. et al., “The Discovery of Nongenotoxic |
| | | Activators of p53: Building on a High-Throughput |
| | | Screen,” Seminars in Canc Bio, 20: 40-45 (2010); |
| | | Wang, Z. and Sun, Y., “Targeting p53 for Novel |
| | | Anticancer Therapy,” Trans Onc, 3: 1-12 (2010); |
| | | US 2011/0021529 |
| Aminothiosl (WR- | Increase of p53 | Mandinova, A. and Lee, S., “The p53 Pathway as a |
| 1065) | activity | Target in Cancer Therapeutics: Obstacles and |
| | | Promise,” Sci Trans Med, 3: 1-7 (2011) |
| Oxindoles | Inhibition of p53- | Weber, L., “Patented inhibitors of p53-Mdm2 |
| | Mdm2 interaction | interaction (2006-2008),” Expert Opin Ther Pat, |
| | | 20(2): 179-91 (2010); U.S. Pat. No. 7,576,082 |
| Spiroindolinones | Inhibition of p53- | Weber, L., “Patented inhibitors of p53-Mdm2 |
| | Mdm2 interaction | interaction (2006-2008),” Expert Opin Ther Pat, |
| | | 20(2): 179-91 (2010); U.S. Pat. No. 6,916,833; |
| | | U.S. Pat. No. 7,495,007; U.S. Pat. No. 7,638,548 |
| Isoquinolines | Inhibition of p53- | Weber, L., “Patented Inhibitors of p53-Mdm2 |
| | Mdm2 interaction | Interaction (2006-2008),” Expert Opin Ther Pat, |
| | | 20(2): 179-91 (2010); U.S. 2009/0306130 |
| Bisaryl sulfonamides | Inhibition of p53- | Weber, L., “Patented Inhibitors of p53-Mdm2 |
| | Mdm2 interaction | Interaction (2006-2008),” Expert Opin Ther Pat, |
| | | 20(2): 179-91 (2010) |
| Substituted piperidines | Inhibition of p53- | U.S. Pat. No. 7,060,713; U.S. Pat. No. 7,553,833 |
| | Mdm2 interaction | |
| Diphenyl-dihydro- | Inhibition of p53- | U.S. Pat. No. 7,625,895 |
| imidazopyridinones | Mdm2 interaction | |
| imidazothiazole | Inhibition of p53- | U.S. 2009/0312310 |
| | Mdm2 interaction | |
| Deazaflavin | Inhibition of p53- | U.S. 2006/0211718; U.S. 2010/0048593 |
| | Mdm2 interaction | |
| Isoindolin-1-one | Inhibition of p53- | U.S. 2008/0261917; U.S. 2011/0224274 |
| | Mdm2 interaction | |
| Boronic acid | Inhibition of p53- | U.S. 2009/0227542; U.S. 2008/0171723 |
| | Mdm2 interaction | |
| Peptides | Inhibition of p53- | U.S. Pat. No. 7,083,983; U.S. 2006/0211757; |
| | Mdm2 interaction | U.S. 2005/0137137; U.S. 2002/0132977; |
| | | U.S. 2009/0030181; U.S. 2010/0168388; |
| | | U.S. 2011/0183917; WO 2008/106507 |
| Pyrrolidin-2-ones | Binding to Mdm2 | U.S. 2010/0075949 |
| Tryptamine (JNJ- | Inhibition of p53- | Yuan Y., et al., “Novel Targeted Therapeutics: |
| 26854165) | Mdm2 interaction | Inhibitors of MDM2, ALK and PARP,” J. Hemat. |
| | | Onc., 4: 16 (2011) |

As used herein, the term “pro-apoptotic agent” refers to any agent, natural or synthetic, that activates the mitochondrial apoptotic cascade to render hepatocytes more resistant to Plasmodium parasite infection or drug-resistant Plasmodium infection, to inhibit the development of Plasmodium parasite liver-stages or drug-resistant Plasmodium parasite liver-stages, to promote apoptosis of Plasmodium-infected hepatocytes or drug-resistant Plasmodium-infected hepatocytes, and/or to sensitize the Plasmodium parasite liver-stages or drug-resistant Plasmodium parasite liver-stages to other, apoptosis-independent treatment. In some embodiments, the pro-apoptotic agent inhibits the action, function, and/or expression of a Bcl-2 family protein. In some embodiments, the pro-apoptotic agent inhibits the action, function, and/or expression of two or more Bcl-2 family member proteins. In some embodiments, the pro-apoptotic agent inhibits the action, function, and/or expression of Bcl-2, Bcl-xL, Mcl-1, or any homolog thereof. In a specific embodiment, the Bcl-2 family protein is Bcl-xL, or any homolog thereof. In some embodiments, the pro-apoptotic agent is a molecule that inhibits, directly or indirectly, the binding of one or more BH3 domains of an anti-apoptotic Bcl-2 family protein to a pro-apoptotic protein, such as from the related second subgroup as described above. In other embodiments, the pro-apoptotic agent increases expression of one or more members of the second or third subgroups related to the Bcl-2 family described above. Briefly, the second and third subgroups related to the Bcl-2 family are pro-apoptotic (unlike the Bcl-2 family itself, which is anti-apoptotic). By increasing expression levels of the second and third related subgroups, the apoptotic pathway can be favored. In further embodiments of the invention, the pro-apoptotic agent can be any agent that targets an upstream or downstream modulator of a BH3 domain-containing protein, for example, by changing the localization, protein level, degradation of, or post-translational modification of a BH3 domain-containing protein.

In some embodiments of the invention, the pro-apoptotic agent is a small drug-like molecule, such as a small molecule mimetic of the BH3 domain. In other embodiments, the pro-apoptotic agent is a peptide, antibody, microRNA, antisense molecule, or other macromolecule. Exemplary pro-apoptotic agents suitable for use in the methods of the present invention have been previously described and some of these have entered clinical trials for the treatment of cancer. Examples of pro-apoptotic agents useful in the practice of the invention include, but are not limited to:

(1) molecules affecting expression of Bcl-2 family members, such as histone deacetylase inhibitors (e.g., sodium butyrate, depsipeptide); synthetic retinoids (e.g., fenretinide), cyclin-dependent kinase inhibitors (e.g., favopiridol), and antisense molecules such as oblimeren sodium; as well as analogs or derivatives of any of the above (Kang, M. H. & Reynolds, C. P., “Bcl-2 Inhibitors: Targeting Mitochondrial Apoptotic Pathways in Cancer Therapy,” Clin Cancer Res 15(4):1126-32 (2009); Azmi, A. S., et al., “Emerging Bcl-2 Inhibitors for the Treatment of Cancer,” Expert Opin Emerg Drugs 16(1):59-70 (2011); Bajwa, N., et al., “Inhibitors of the Anti-Apoptotic Bcl-2 Proteins: A Patent Review” Expert Opin Ther Pat 22(1):37-55 (2012), each incorporated herein by reference in its entirety);

(2) antibody, ribozyme, and peptide inhibitors (such as BH3 peptides); and analogs and derivatives of any of the above (see, e.g., Bajwa, N., et al., “Inhibitors of the anti-apoptotic Bcl-2 proteins: a patent review” Expert Opin Ther Pat 22(1):37-55 (2012); Azmi, A. S., et al., “Emerging Bcl-2 inhibitors for the treatment of cancer,” Expert Opin Emerg Drugs 16(1):59-70 (2011); U.S. Pat. No. 7,354,928; WO9704006; WO9916787; WO2004058804; WO2006000034; WO2005044839; U.S. Pat. No. 7,723,469, each incorporated herein by reference in its entirety);

(3) small molecule inhibitors, such as natural compounds extracted from teas and synthetic versions thereof (e.g. gossypol); inhibitors represented by ABT-737, ABT-263, indole bipyrrole compounds (e.g., GX15-070), inhibitors represented by HA14-1; inhibitors represented by BH3 inhibitors 1 and 2 and antimycin; and inhibitors represented by obatoclax; isoxazolidine derived inhibitors; and benzoyl urea derived inhibitors; inhibitors represented by AT-101; inhibitors represented by TW-37; as well as analogs or derivatives of any of the above (Kang, M. H. & Reynolds, C. P., “Bcl-2 Inhibitors: Targeting Mitochondrial Apoptotic Pathways in Cancer Therapy,” Clin Cancer Res 15(4):1126-32 (2009); Bajwa, N., et al., “Inhibitors of the Anti-Apoptotic Bcl-2 Proteins: A Patent Review” Expert Opin Ther Pat 22(1):37-55 (2012); U.S. Pat. No. 7,812,058; WO2002097053; U.S. Pat. No. 7,432,304; WO2005069771; WO2005094804; U.S. Pat. No. 7,342,046; U.S. Pat. No. 7,432,300; WO2006050447; WO2009052443; U.S. Pat. No. 8,039,668; WO2010120943; WO2006023778; WO2004106328; WO2005117908; U.S. Pat. No. 7,425,553; U.S. Pat. No. 7,642,260; US20070072860; U.S. Pat. No. 7,973,161; WO2002024636; WO2005049593; WO2005049594; U.S. Pat. No. 7,767,684; U.S. Pat. No. 7,906,505; WO2006127364; U.S. Pat. No. 7,777,076; WO2005117543; U.S. Pat. No. 7,585,858; WO2009155386; WO2010083442; WO2010065865; WO2008130970; U.S. Pat. No. 7,981,888; WO2008131000; WO2002060887; U.S. Pat. No. 6,660,871; WO2000114365; U.S. Pat. No. 7,241,804; WO2008060569; U.S. Pat. No. 7,842,815; U.S. Pat. No. 7,851,637; WO2006002474; U.S. Pat. No. 7,956,216; each of which is incorporated herein by reference in its entirety).

The pro-apoptotic agent and p53 activator, such as the non-limiting examples discussed hereinabove, can be used in any combination in the context of the illustrative methods and embodiments described herein. Illustrative, non-limiting examples of combinations of pro-apoptotic agents and p53 activators for administration in any of the described methods include use of Serdementan as a p53 activator and Obataclax, ABT-737, ABT-199, and/or any other pro-apoptotic agent contemplated herein. Another example is use of Nutlin-3 as a p53 activator and Obataclax, ABT-737, ABT-199, and/or any other pro-apoptotic agent contemplated herein.

Accordingly, one aspect of the invention provides methods for preventing infection of hepatocytes by a Plasmodium parasite or a drug-resistant Plasmodium parasite, which comprise the step of contacting a hepatocyte with amounts of both a pro-apoptotic agent and a p53 activator effective to prevent infection of a hepatocyte by a Plasmodium parasite or a drug-resistant Plasmodium parasite. As used herein, the term “prevent infection of a hepatocyte” refers to averting or inhibiting the entry of the parasite or drug-resistant parasite into a susceptible hepatocyte, or, alternatively, preventing or inhibiting survival of the parasite upon contact with or entry into a susceptible hepatocyte. The hepatocyte may be contacted with a pro-apoptotic agent and a p53 activator prior to, concurrently with, or subsequent to exposure to the Plasmodium parasite or a drug-resistant Plasmodium parasite. Moreover, the hepatocyte may be contacted with a pro-apoptotic agent prior to, concurrently with or subsequent to the hepatocyte being contacted with a p53 activator. In some embodiments, the hepatocyte is contacted with both a pro-apoptotic agent and p53 activator in vitro, as described in more detail below. Appropriate effective amounts of the pro-apoptotic agent and p53 activator with which to contact hepatocytes in vitro may be readily determined using only routine experimentation (see, e.g., the disclosure provided below). In some embodiments, the hepatocyte is contacted with both a pro-apoptotic agent and a p53 activator in vivo, as described further below.

Another aspect of the invention provides methods for inhibiting the growth or development of liver stage Plasmodium parasites or drug-resistant Plasmodium parasites, which comprise the step of contacting Plasmodium liver stage parasites or drug-resistant liver stage Plasmodium parasites with amounts of both a pro-apoptotic agent and a p53 activator effective to inhibit the growth of liver stage Plasmodium parasites or drug-resistant liver stage Plasmodium parasites. The term “inhibit the growth or development of liver stage Plasmodium parasites or drug-resistant liver stage Plasmodium parasites” refers to preventing, slowing, suppressing or otherwise interfering with the growth or development of Plasmodium liver stage parasites or drug-resistant liver stage Plasmodium parasites, including, for example, by killing liver stage parasites or drug-resistant liver stage parasites. The term also encompasses causing the arrest or alternation of typical development such that fewer or no viable merezoites are produced and there is a reduction or elimination of blood stage parasites that can infect erythrocytes. In some embodiments, the invention provides methods for inhibiting the growth or development of P. vivax or P. ovale hypnozoites, including drug-resistant P. vivax or P. ovale hypnozoites. In some embodiments, the Plasmodium liver stage parasites or drug-resistant liver stage parasites are contacted with both a pro-apoptotic agent and a p53 activator in vitro, as described in more detail below. Appropriate effective amounts of the pro-apoptotic agent and the p53 activator with which to contact liver stage parasites in vitro may be readily determined using only routine experimentation (see, e.g., the disclosure provided below). In some embodiments, the Plasmodium liver stage parasites or drug-resistant liver stage parasites are contacted with both a pro-apoptotic agent and a p53 activator in vivo, as described further below.

A further aspect of the invention provides methods for preventing infection of hepatocytes by a Plasmodium parasite or a drug-resistant parasite in a vertebrate subject. These methods comprise the step of administering to a vertebrate subject in need thereof amounts of both a pro-apoptotic agent and a p53 activator effective to prevent infection by a Plasmodium parasite or a drug-resistant parasite of hepatocytes in the vertebrate subject. The pro-apoptotic agent and/or the p53 activator may be administered prior to, concurrently with, or subsequent to the exposure of the vertebrate subject to a Plasmodium parasite or a drug-resistant parasite. Moreover, the hepatocyte may be contacted with a pro-apoptotic agent prior to, concurrently with, or subsequent to the hepatocyte being contacted with a p53 activator. In some embodiments, the Plasmodium parasite or drug-resistant parasite is P. falciparum, P. vivax, or P. ovale. In some embodiments, the vertebrate subject, such as a human subject, is administered the pro-apoptotic agent and/or the p53 activator prior to potential or anticipated exposure to potentially Plasmodium-bearing Anopheles mosquito vectors, such as at least 6 hours, 12 hours, 24 hours, 36 hours, 48 hours, or more, or any subrange therein prior to exposure to potentially Plasmodium-bearing Anopheles mosquito vectors.

Yet another aspect of the invention provides methods for inhibiting the growth or development of liver stage Plasmodium parasites or drug-resistant Plasmodium parasites in a vertebrate subject, comprising the step of administering to a vertebrate subject in need thereof amounts of both a pro-apoptotic agent and a p53 activator effective to inhibit the growth of liver stage parasites or drug-resistant liver stage parasites. The pro-apoptotic agent and/or the p53 activator may be administered prior to, concurrently with, or subsequent to the exposure of the vertebrate subject to a Plasmodium parasite or a drug-resistant parasite. Moreover, the pro-apoptotic agent may be administered to the vertebrate subject prior to, concurrently with, or subsequent to the administration of the p53 activator. In some embodiments, the Plasmodium parasite or drug-resistant parasite is P. falciparum, P. vivax, or P. ovale. In some embodiments, the parasite or drug-resistant parasite is a P. vivax or P. ovale hypnozoite. The vertebrate subjects in need include those subjects already infected with Plasmodium parasites or drug-resistant parasites (such as liver stage parasites) and those who are at risk of being infected with Plasmodium parasites or drug-resistant parasites.

Another aspect of the invention provides a method for treating a vertebrate subject suffering from a Plasmodium parasite infection or a drug-resistant parasite infection, comprising the step of administering to a vertebrate subject suffering from a Plasmodium parasite infection or a drug-resistant parasite infection effective amounts of both a pro-apoptotic agent and a p53 activator. The pro-apoptotic agent and/or the p53 activator may be administered prior to, concurrently with, or subsequent to the exposure of the vertebrate subject to a Plasmodium parasite or a drug-resistant parasite. Moreover, the pro-apoptotic agent may be administered to the vertebrate subject prior to, concurrently with, or subsequent to the administration of the p53 activator. The term “treating” refers to both therapeutic treatment and prophylactic or preventative measures, wherein the object is to prevent or slow down (lessen) the targeted pathologic condition or disease. Treatment includes radical cure of P. vivax infection, eliminating hypnozoites that can cause relapsing disease after initial infection. Those in need of treatment include those already with the disease as well as those prone to have the disease or those in whom the disease is to be prevented. Accordingly, in some embodiments, the subjects to be treated are human subjects suffering from malaria caused by a Plasmodium parasite or a drug-resistant Plasmodium parasite. In other embodiments, the subjects to be treated are human subjects at risk for contracting malaria caused by a Plasmodium parasite or a drug-resistant Plasmodium parasite.

In some embodiments, the Plasmodium parasite infection or drug-resistant Plasmodium parasite infection is a liver stage infection and the amounts of the pro-apoptotic agent and the p53 activator administered is effective to treat the liver stage infection. The methods of the invention also encompass treating mixed Plasmodium infections, such as a mixed P. falciparum and P. vivax infection, or mixed drug-resistant Plasmodium parasite infections such as a mixed drug-resistant P. falciparum and drug-resistant P. vivax infection. In some embodiments, the liver stage infection or drug-resistant liver stage infection is a dormant infection, such as caused by a dormant infection by P. vivax and P. ovale hypnozoites. In some embodiments, the methods of the invention prevent the relapse of P. vivax and P. ovale infections by eradicating hypnozoites.

Another aspect of the invention provides methods for preventing or treating malaria caused by a Plasmodium parasite or a drug-resistant Plasmodium parasite in a vertebrate subject, comprising the step of administering to a vertebrate subject in need thereof amounts of both a pro-apoptotic agent and a p53 activator effective to prevent malaria. The vertebrate subjects in need include those subjects already infected with Plasmodium parasites or drug-resistant Plasmodium parasites (such as liver stage parasites) and those subjects who are at risk of being infected with Plasmodium parasites or drug-resistant Plasmodium parasites. The term “preventing malaria” refers to averting the clinical manifestations of blood stage malaria resulting from the infection of erythrocytes with merozoites, for example, by preventing hepatocyte infection or inhibiting the growth or development of liver stages such that insufficient (including none) or inviable merezoites result from the liver stage. The liver stage of the Plasmodium parasite is clinically silent and precedes the blood stage infection. Destroying the liver stage parasite would thus prevent the onset of disease. In some embodiments, the methods of the invention prevent the relapse of P. vivax and P. ovale infections by eradicating hypnozoites. The pro-apoptotic agent and/or the p53 activator may be administered prior to, concurrently with, or subsequent to the exposure of the vertebrate subject to a Plasmodium parasite or drug-resistant Plasmodium parasite. Moreover, the pro-apoptotic agent may be administered to the vertebrate subject prior to, concurrently with, or subsequent to the administration of the p53 activator. In some embodiments, the pro-apoptotic agent and p53 activator are administered prior to the appearance of blood stage Plasmodium parasites or drug-resistant parasites in amounts effective to prevent infection of hepatocytes by Plasmodium parasites or drug-resistant parasites. In some embodiments, the pro-apoptotic agent and p53 activator are administered prior to the appearance of blood stage Plasmodium parasites or drug-resistant parasites in amounts effective to inhibit the growth of liver stage Plasmodium parasites or drug-resistant parasites in the subject. In some embodiments, the Plasmodium parasites or drug-resistant parasites are one of P. falciparum, P. vivax, and P. ovale parasites.

A further aspect of the invention provides methods for eliciting protective immunity against malaria caused by a Plasmodium parasite or a drug-resistant Plasmodium parasite in a vertebrate subject, comprising the step of administering to a vertebrate subject an amount of either a pro-apoptotic agent or a p53 activator, or amounts of both a pro-apoptotic agent and a p53 activator, effective to elicit protective immunity against malaria in the vertebrate subject.

As used herein, the term “eliciting protective immunity against malaria” refers to enhancing the host immune response to parasites by preventing the parasite from proceeding to blood stage and causing malarial disease. It has been demonstrated that sterile protective immunity is achieved after vaccination with GAPs in the rodent malaria models. In addition, an Infection-Treatment Vaccination (ITV) study in human subjects has shown that controlling liver stage development of Plasmodium parasites by treating the human subject with suppressive prophylaxis resulted in complete protection upon subsequent challenge with homologous parasites (Roestenberg, M., et al., “Protection Against a Malaria Challenge by Sporozoite Inoculation,” N Engl J Med. 361:468-477 (2009), incorporated herein by reference in its entirety). This ITV strategy prevents blood-stage infection but allows exposure and immune sensitization to liver-stage parasites and antigens. A similar effect may be obtained by inhibiting the growth or development of liver-stage parasites in a vertebrate subject by treating the subject with both a pro-apoptotic agent and p53 activator. Therefore, in some embodiments of the invention, a pro-apoptotic agent and p53 activator can be used to induce protective immunity in the vertebrate subject by administering the pro-apoptotic agent and p53 activator to a subject that is infected with Plasmodium LS or will be exposed to Plasmodium challenge.

In another aspect, the disclosure provides compositions for use in treating a vertebrate subject with a Plasmodium parasite infection or a drug-resistant parasite infection, for use in preventing or treating malaria caused by a Plasmodium parasite or a drug-resistant Plasmodium parasite in a vertebrate subject, and for eliciting protective immunity against malaria caused by a Plasmodium parasite or a drug-resistant Plasmodium parasite in a vertebrate subject, as described above. The compositions according to this aspect comprise an effective amount of at least one pro-apoptotic agent, as described herein, and an effective amounts at least one p53 activator, as described herein. The effective amounts of the at least one pro-apoptotic agent can be administered prior to, concurrently with, or subsequent to the administration of the at least one p53 activator. Furthermore, the pro-apoptotic agent and/or the p53 activator can be administered prior to, concurrently with, or subsequent to the exposure of the vertebrate subject to a Plasmodium parasite or drug-resistant Plasmodium parasite. In some embodiments, the pro-apoptotic agent and/or p53 activator inhibit the growth of Plasmodium liver stage parasites or drug-resistant Plasmodium liver stage parasites. In some embodiments, the Plasmodium liver stage parasites fail to produce blood stage merezoites after administration of the pro-apoptotic agent and p53 activator, thus resulting in a prevention or reduction in malaria disease. In some embodiments, the subject develops at least some degree of protective immunity against future infections by the Plasmodium parasite. In some embodiments, the Plasmodium parasite or drug-resistant parasite is P. falciparum or P. vivax. In some embodiments, the vertebrate subject is a mammal, such as a primate or rodent. Exemplary subjects include humans, mice, and rats.

The pro-apoptotic agent and the p53 activator may be administered to a subject in any suitable pharmaceutical composition(s) or formulation(s) suitable for oral, topical, parenteral application, or the like. The pro-apoptotic agent and the p53 activator may be combined in one composition or formulation or may be contained in separate compositions or formulations. The composition(s) or formulation(s) of the invention may include a pharmaceutically acceptable carrier. Any dosage forms may be selected depending on purpose, as is well-understood by persons of ordinary skill in the art. Exemplary pro-apoptotic agent compositions and formulations and methods of administrations suitable for use in the methods of the present invention have been previously described, for example, in Bedikian et al., “Bcl-2 Antisense (oblimersen sodium) Plus Dacarbazine in Patients With Advanced Melanoma: The Oblimersen Melanoma Study Group,” J Clin Oncol 24:4738-4745 (2006); U.S. Pat. No. 7,812,058, U.S. Pat. No. 7,354,928; WO9704006; WO9916787; WO2004058804; WO2006000034; WO2005044839; U.S. Pat. No. 7,723,469; U.S. Pat. No. 7,812,058; WO2002097053; U.S. Pat. No. 7,432,304; WO2005069771; WO2005094804; U.S. Pat. No. 7,342,046; U.S. Pat. No. 7,432,300; WO2006050447; WO2009052443; U.S. Pat. No. 8,039,668; WO2010120943; WO2006023778; WO2004106328; WO2005117908; U.S. Pat. No. 7,425,553; U.S. Pat. No. 7,642,260; US20070072860; U.S. Pat. No. 7,973,161; WO2002024636; WO2005049593; WO2005049594; U.S. Pat. No. 7,767,684; U.S. Pat. No. 7,906,505; WO2006127364; U.S. Pat. No. 7,777,076; WO2005117543; U.S. Pat. No. 7,585,858; WO2009155386; WO2010083442; WO2010065865; WO2008130970; U.S. Pat. No. 7,981,888; WO2008131000; WO2002060887; U.S. Pat. No. 6,660,871; WO2000114365; U.S. Pat. No. 7,241,804; WO2008060569; U.S. Pat. No. 7,842,815; U.S. Pat. No. 7,851,637; WO2006002474; U.S. Pat. No. 7,956,216; each of which is hereby incorporated by reference in its entirety. Exemplary p53 activator compositions and formulations and methods of administrations suitable for use in the methods of the present invention have been previously described, for example, in U.S. Pat. No. 7,759,383, U.S. Patent Publication Nos. 20050008653, 20050227932, 20100137345, and 20100143332, each of which is hereby incorporated by reference in its entirety.

The term “effective amount” for a therapeutic or prophylactic treatment refers to an amount or dosage of a composition sufficient to induce a desired response or outcome (e.g., prevention of parasite infection or alleviation of malaria symptoms) in subjects to which it is administered. The effective amount and method of administration of a particular therapeutic or prophylactic treatment may vary based on the individual subject and the stage of the disease, as well as other factors known to those of skill in the art. Therapeutic efficacy and toxicity of such compounds may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies are used in formulating a range of dosages for human use. The dosage of such compounds lies preferably within a range of circulating concentrations that include the ED50 with little or no toxicity. The dosage varies within this range depending upon the dosage form employed, sensitivity of the subject, and the route of administration. The exact dosage is chosen by the individual physician in view of the subject to be treated. Dosage and administration are adjusted to provide sufficient levels of the active moieties or to maintain the desired effect. Additional factors that may be taken into account include the prevalence of the Plasmodium parasite or drug-resistant parasite, such as, for example, P. falciparum, in the geographical vicinity of the subject, the severity of the disease, the degree of resistance of the parasite to standard drug treatments, state of the patient, age, and weight of the subject, diet, time and frequency of administration, drug combination(s), reaction sensitivities, and tolerance/response to therapy. An appropriate effective amount may be readily determined using only routine experimentation. Several doses may be needed per subject in order to achieve a sufficient response to effect treatment.

In some embodiments, an effective amount of each of the pro-apoptotic agent and the p53 activator is between from about 0.001 to 500 mg/kg daily. For example, from about 1 micrograms/kg to 50 milligrams/kg daily, or between about 5 micrograms/kg and 10 micrograms/kg daily, or between about 10 to 20 micrograms/kg daily, or between about 20 to about 25 micrograms/kg daily, or between about 25 to 50 micrograms/kg daily, or between about 50 to 100 micrograms/kg daily, or between about 100 to 500 micrograms/kg daily, depending on the age, height, sex, general medical condition, previous medical history, as well as other factors known to those of skill in the art. In some embodiments, an effective amount is between 1-3500 micrograms/kg weekly, such as 5-50 micrograms/kg weekly, or 200-1000 micrograms/kg weekly, or 1000-3500 micrograms/kg weekly, depending on the age, height, sex, general medical condition, previous medical history, as well as other factors known to those of skill in the art.

A person of skill in the art will readily appreciate that all aspects of the disclosure apply equally to a drug-resistant Plasmodium parasite as to a Plasmodium parasite.

It is generally noted that the use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense as opposed to an exclusive or exhaustive sense, such as in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. Words such as “about” and “approximately” imply minor variation around the stated value, usually within a standard margin of error, such as within 10% or 5% of the stated value.

Disclosed are materials, compositions, and components that can be used for, in conjunction with, and in preparation for the disclosed methods and compositions. It is understood that when combinations, subsets, interactions, groups, etc., of these materials are disclosed each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following is a description of the surprising discovery that the combination of Bcl2-inhibitors and p53 agonists results in a greatly enhanced reduction, and often complete elimination, of liver stage Plasmodium parasites from host cells in vitro and in vivo. These results demonstrate that a combined therapy of Bcl2-inhibitors and p53 agonists is useful for host-based clearance and prophylactic treatment of malaria.

Abstract/Rationale

Over three billion people in 106 countries are at risk for malaria, which kills approximately one million annually, primarily pregnant women and children. When the infectious form of the Plasmodium parasite is transmitted via the bite of an infected Anopheles mosquito, it rapidly travels to the liver where it rapidly divides over the next 2-8 days.

Eliminating malaria parasites during the asymptomatic but obligate liver stages of infection would stop disease and subsequent transmission. Unfortunately, only a single licensed drug that targets all liver stages, Primaquine, is available. While drugs like Primaquine, exist that target liver stage parasites, drug resistance and side-effects limit their use. Targeting host proteins might significantly expand the repertoire of prophylactic drugs against malaria. Recently, we have found that malaria-infected hepatocytes share some qualities with cancer cells, namely that the Bcl-2 oncogene is elevated and the tumor suppressor p53 is dampened. Here, it is demonstrated that both Bcl-2 inhibitors and p53 agonists dramatically reduce liver stage burden in a mouse malaria model in vitro and in vivo by altering the activity of key hepatocyte factors upon which the parasite relies. Bcl-2 inhibitors act primarily by inducing apoptosis in infected hepatocytes, whereas p53 agonists eliminate parasites in an apoptosis-independent fashion. Significantly, in combination Bcl-2 inhibitors and p53 agonists act synergistically to delay, and in some cases completely prevent, the onset of blood stage disease in mice. Both families of drugs are highly effective at doses that do not cause substantial hepatocyte cell death in vitro or liver damage in vivo. When administered to humanized mice infected with P. falciparum, pre-erythrocytic parasites were also substantially reduced. These data demonstrate that host-based prophylaxis is an effective intervention strategy to eliminate liver stage parasites before the onset of clinical disease and, thus, opens a new avenue to treat and prevent malaria.

### INTRODUCTION

Plasmodium parasites cause malaria worldwide, infecting 200-500 million and killing nearly over 600,000 people annually. Despite the impact of the disease and efforts over decades to eradicate it, malaria persists worldwide. One of the roadblocks to eradication has been the development of drug-resistant parasites, which often evolve within years of the distribution of new anti-malarial drugs. All currently available treatments and prophylactic regimens are thought to directly target parasite proteins. However, their rapid replication allows parasites to quickly develop mutations that render them resistant to treatment. While combination therapies based on artemisinin have recently been more effective at circumventing the development of drug resistance, this strategy is beginning to lose potency as the parasite develops resistance to each drug.

The complex lifecycle of the malaria parasite provides multiple potential points for intervention. Plasmodium parasites are deposited in the skin by the bite of a female Anopheles mosquito before they travel to the liver. Once in the liver, parasites traverse the sinusoids, enter the parenchyma, and invade hepatocytes. Over the next 2-10 days, the liver stage (LS) parasite exploits the resources of its host hepatocyte to produce tens of thousands of red blood cell-infectious progeny. While parasites divide more quickly within the hepatocyte than any other time in their lifecycle, symptomatic disease is only initiated after the liver stage is complete and the erythrocytic stage begins. The liver also harbors long-lived dormant forms of Plasmodium vivax called hypnozoites, which are the source of relapsing infection (White, N. J., “Determinants of Relapse Periodicity in Plasmodium vivax Malaria,” Malar J 10:297 (2011), incorporated herein by reference in its entirety). Eliminating the liver stage parasite would prevent initial and relapsing disease and subsequent transmission. Yet there is only a single licensed drug, Primaquine, that targets all LS parasites, and its use is limited by side-effects.

The LS parasite relies on a precise intracellular environment that supports growth, as evident in part by the minimal development of axenic parasite culture (Gangoso, E., et al., “A Cell-Penetrating Peptide Based on the Interaction Between c-Src and connexin43 Reverses Glioma Stem Cell Phenotype,” Cell Death & Disease 5:e1023 (2014), incorporated herein by reference in its entirety). Thus, even slight perturbations of key hepatocyte factors using host-based prophylactic (HBP) drugs might completely prevent the parasite from proceeding to blood stage disease. The inventors have previously shown that Plasmodium parasites manipulate several hepatocyte factors involved in cell survival signaling during LS infection (Kaushansky, A., et al., “Suppression of Host p53 is Critical for Plasmodium Liver-Stage Infection,” Cell Reports 3:630-637 (2013); Albuquerque, S. S., et al., “Host Cell Transcriptional Profiling During Malaria Liver Stage Infection Reveals a Coordinated and Sequential Set of Biological Events,” BMC Genomics 10:270 (2009), each incorporated by reference in its entirety). Specifically, parasites actively suppress the tumor suppressor p53 (Kaushansky, A., et al., Cell Reports 3:630-637 (2013), which is involved in a variety of cellular outcomes including apoptosis and the cell cycle arrest (Li, T., et al., “Tumor Suppression in the Absence of p53-Mediated Cell-Cycle Arrest, Apoptosis, and Senescence,” Cell 149:1269-1283 (2012), incorporated herein by reference in its entirety). Malaria parasites also modulate the mitochondrial apoptotic cascade by increasing levels of the pro-survival Bcl-2 family members, and by suppressing levels of the pro-apoptotic factor Bad (Kaushansky, A., et al., Cell Reports 3:630-637 (2013). Reversing either parasite-driven change in the hepatocyte reduces liver stage burden, indicating that p53 suppression and Bcl-2 family activity are critical for parasite survival (Kaushansky, A., et al., Cell Reports 3:630-637 (2013); Kaushansky, A., et al., “Malaria Parasite Liver Stages Render Host Hepatocytes Susceptible to Mitochondria-Initiated Apoptosis,” Cell Death & Disease 4:e762 (2013), each incorporated herein by reference in its entirety). Consequently, increasing levels of p53 using genetic or pharmacological approaches reduces liver stage burden (Kaushansky, A., et al., Cell Reports 3:630-637 (2013). Similarly, blocking the Bcl-2 family activity eliminates malaria parasites via hepatocyte apoptosis (Kaushansky, A., et al., Cell Death & Disease 4:e762 (2013)). This disclosure describes investigations of the capacity of these interventions as prophylaxis regimens against rodent and human malarias. Identifying a drug regimen that eliminates liver stage parasites could ease the burden of malaria worldwide.

Results

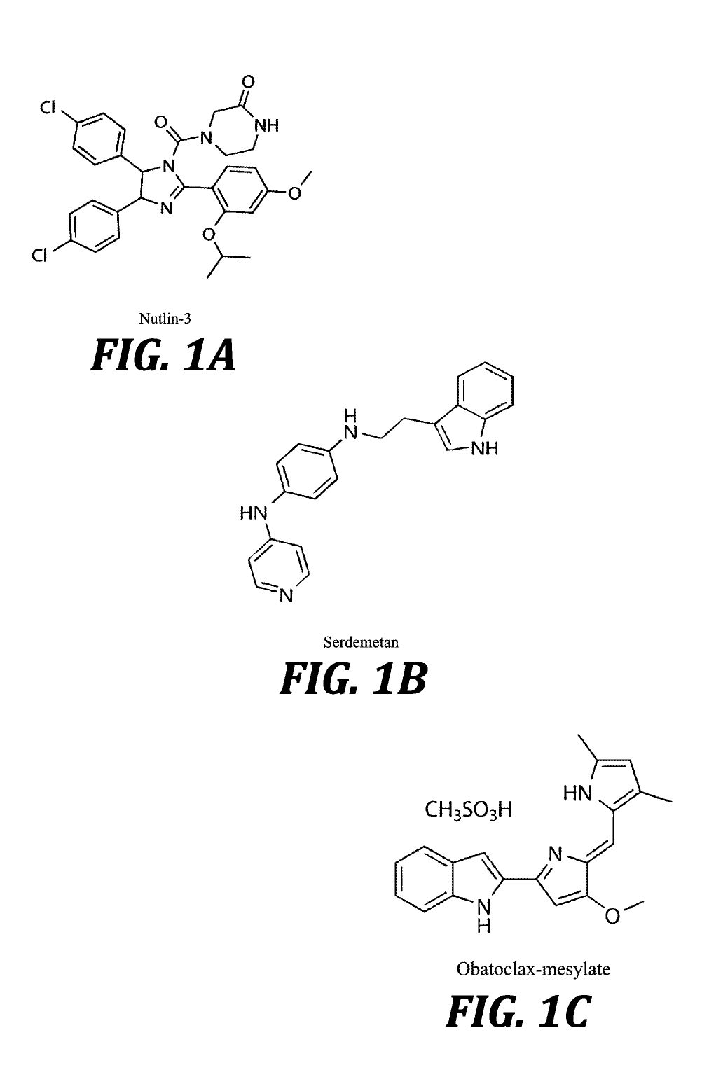

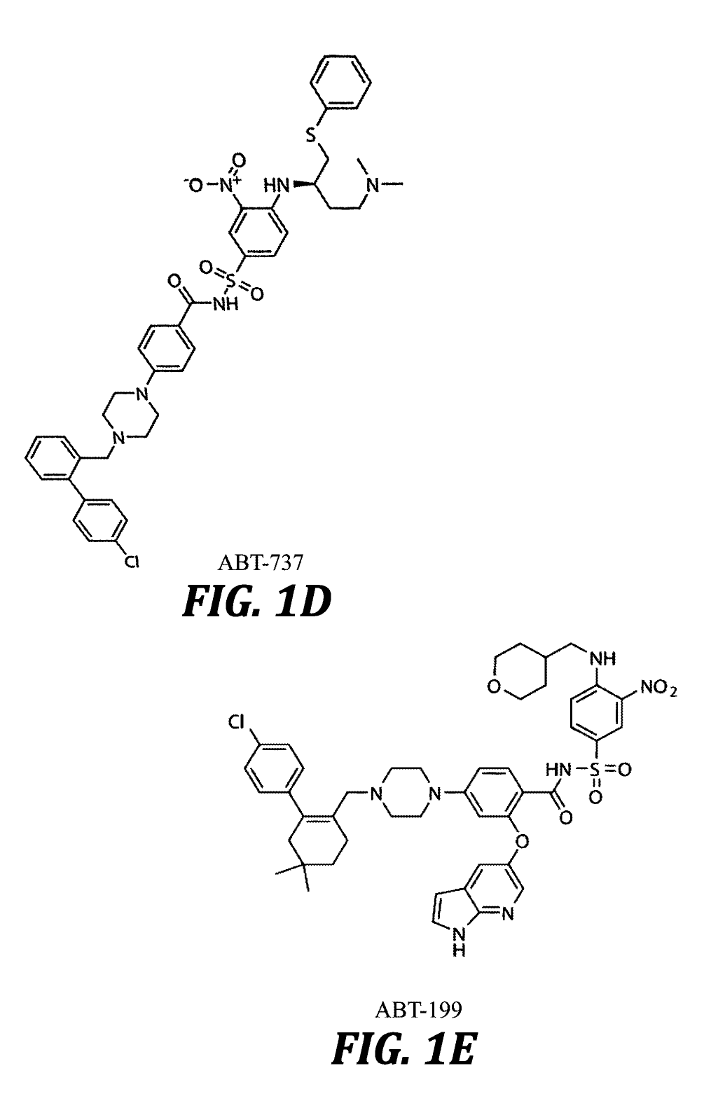

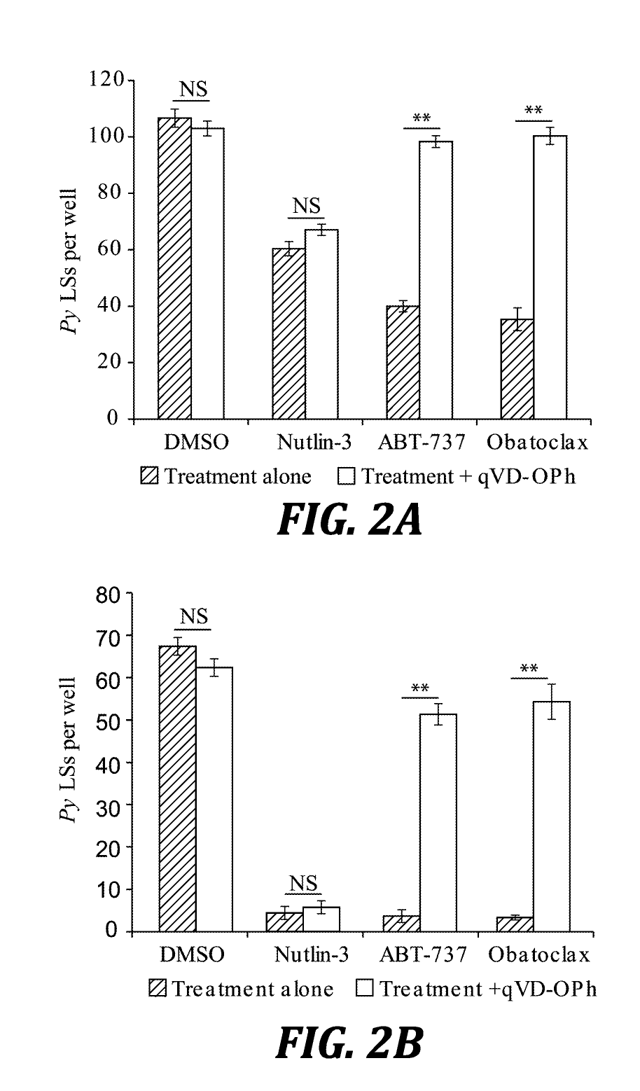

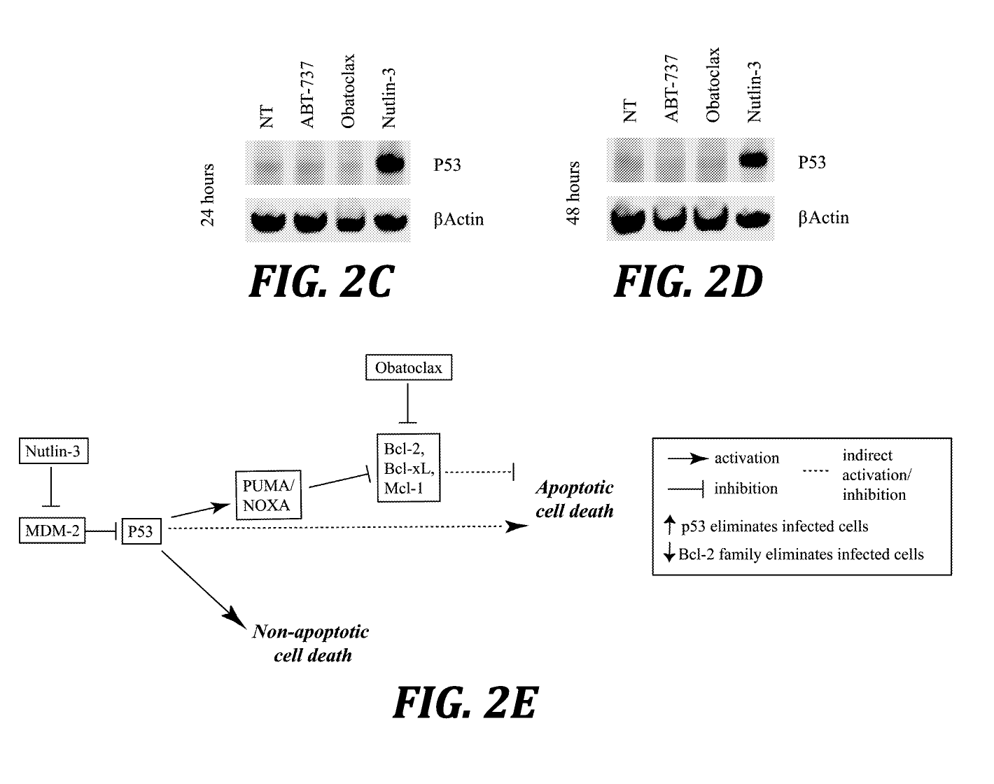

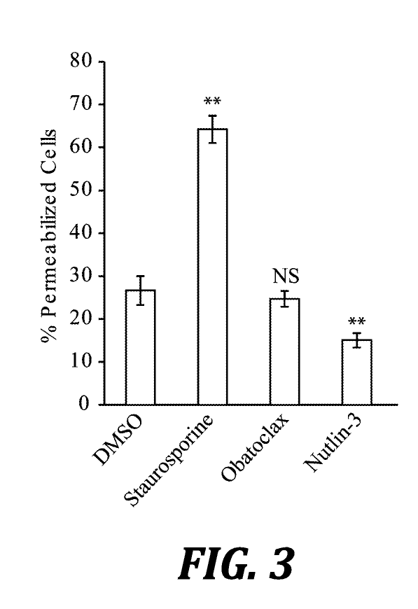

Modulating hepatocyte factors, such as p53 and Bcl-2, that Plasmodium requires for complete liver stage development can efficiently eliminate parasites (Kaushansky, A., et al., Cell Reports 3:630-637 (2013); Kaushansky, A., et al., Cell Death & Disease 4:e762 (2013)), although the mechanism remains unexplored. Several chemotherapeutic agents have been developed and clinically tested that target p53 or Bcl-2 family proteins (Brown, C. J., et al., “Awakening Guardian Angels: Drugging the p53 Pathway,” Nature Reviews: Cancer 9:862-873 (2009); Schimmer, A. D., et al., “A Phase I Study of the pan bcl-2 Family Inhibitor Obatoclax Mesylate in Patients With Advanced Hematologic Malignancies,” Clinical Cancer Research: an official journal of the American Association for Cancer Research 14:8295-8301 (2008), each incorporated herein by reference in its entirety). Nutlin-3 increases p53 levels by binding to the ubiquitin-ligase MDM-2 and preventing p53 degradation (Brown, C. J., et al., Nature Reviews: Cancer 9:862-873 (2009)), whereas Obatoclax and ABT-737 inhibit multiple pro-survival Bcl-2 family proteins (Hartman, M. L. & Czyz, M., “Pro-Apoptotic Activity of BH3-Only Proteins and BH3 Mimetics: From Theory to Potential Cancer Therapy,” Anti-cancer Agents in Medicinal Chemistry 12:966-981 (2012) and Cat. No. S1061, Selleck Chemicals, Houston, Tex., each incorporated herein by reference in its entirety) (see FIGS. 1A, 1C, and 1D, respectively for chemical structures). Both p53 and the Bcl-2 family proteins also have well-described roles in hepatocyte apoptosis. Thus, we asked to what extent apoptosis was responsible for parasite clearance in response to elevated p53 (Nutlin-3) or inhibition of the Bcl-2 family (ABT-737 or Obatoclax). We infected Hepa 1-6 cells with Plasmodium yoelii sporozoites, and then treated with each drug alone or in combination with a pan-caspase inhibitor, qVD-OPh (FIGS. 2A and 2B). qVD-OPh reverses nearly all apoptosis in Hepa 1-6 cells (data not shown). Treatments with ABT-737 or Obatoclax alone reduced LS by 80-85% after either 24 hours (P=0.000033 and P=0.000027, respectively) or 48 hours (P=0.0000044 and P=0.00014, respectively). The addition of qVD-OPh almost completely reversed this effect for both treatments. This indicates that infected hepatocytes treated with Bcl-2 inhibitors are eliminated by apoptosis of the host cell. Strikingly, while Nutlin-3 eliminated 97% of liver stage parasites after 48 hours (P=0.0000046), this effect was completely unaltered by the addition of qVD-OPh. While some apoptotic pathways do involve p53, ABT-737 and Obatoclax do not increase p53 levels in Hepa 1-6 cells (FIGS. 2C and 2D). Importantly, when cell deaths were assessed in the total culture, no increase was observed in uninfected hepatoma cell death following a 48 hour treatment with ABT-737, Obatoclax, or Nutlin-3 (FIG. 3). This demonstrates that infected cells were selectively eliminated without inducing substantial damage to uninfected cells. We have previously demonstrated that none of these drugs target parasites directly (Kaushansky, A., et al., Cell Reports 3:630-637 (2013); Kaushansky, A., et al., Cell Death & Disease 4:e762 (2013)), suggesting that parasite infection renders hepatocytes sensitive to these drugs. Taken together, these data demonstrate that p53 agonists and Bcl-2 family inhibitors both eliminate infected hepatocytes by targeting the host cell, but do so using different pathways (FIG. 2E).

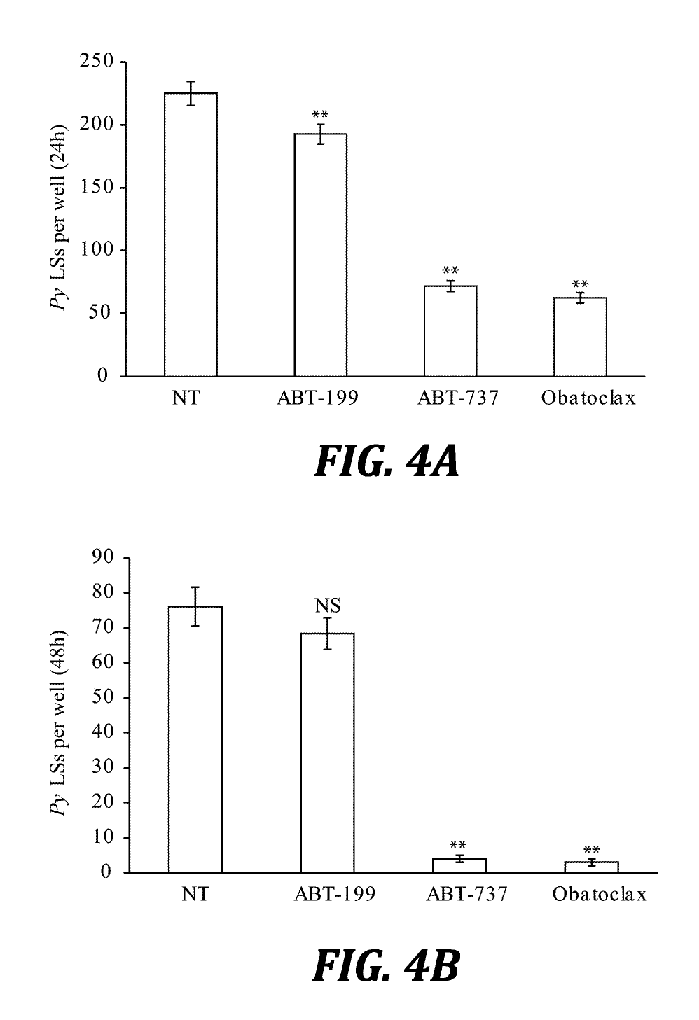

We next asked which pro-survival member of the Bcl-2 family plays the largest role in ensuring the survival of LS-infected hepatocytes. Multiple compounds have been characterized for their activity against pro-survival Bcl-2 family members Bcl-2, Bcl-xL and Mcl-1 (Billard, C., “BH3 Mimetics: Status of the Field and New Developments,” Molecular Cancer Therapeutics 12:1691-1700 (2013), incorporated herein by reference in its entirety). ABT-199 has been demonstrated to inhibit Bcl-2 alone (Wongsrichanalai, C., et al., “Epidemiology of Drug-Resistant Malaria,” The Lancet Infectious Diseases 2:209-218 (2002), incorporated herein by reference in its entirety), ABT-737 inhibits Bcl-2 and Bcl-xL (Oltersdorf, T., et al., “An Inhibitor of Bcl-2 Family Proteins Induces Regression of Solid Tumours,” Nature 435:677-681 (2005), incorporated herein by reference in its entirety), and Obatoclax inhibits Bcl-2, Bcl-xL and Mcl-1 (Cat. No. S1061, Selleck Chemicals, Houston, Tex.; Nguyen, M., et al., “Small Molecule Obatoclax (GX15-070) Antagonizes MCL-1 and Overcomes MCL-1-Mediated Resistance to Apoptosis,” Proceedings of the National Academy of Sciences of the United States of America 104:19512-19517 (2007), incorporated herein by reference in its entirety) (see FIGS. 1A-1E for chemical structures). Interestingly, ABT-199 did not reduce LS burden, suggesting that targeting Bcl-2 alone is insufficient to eliminate infected hepatocytes (FIGS. 4A and 4B). However, inhibiting Bcl-xL in addition to Bcl-2, the primary targets of ABT-737, efficiently eliminates parasites, suggesting that Bcl-xL is the key Bcl-2 family member required for parasite survival during its liver stage.

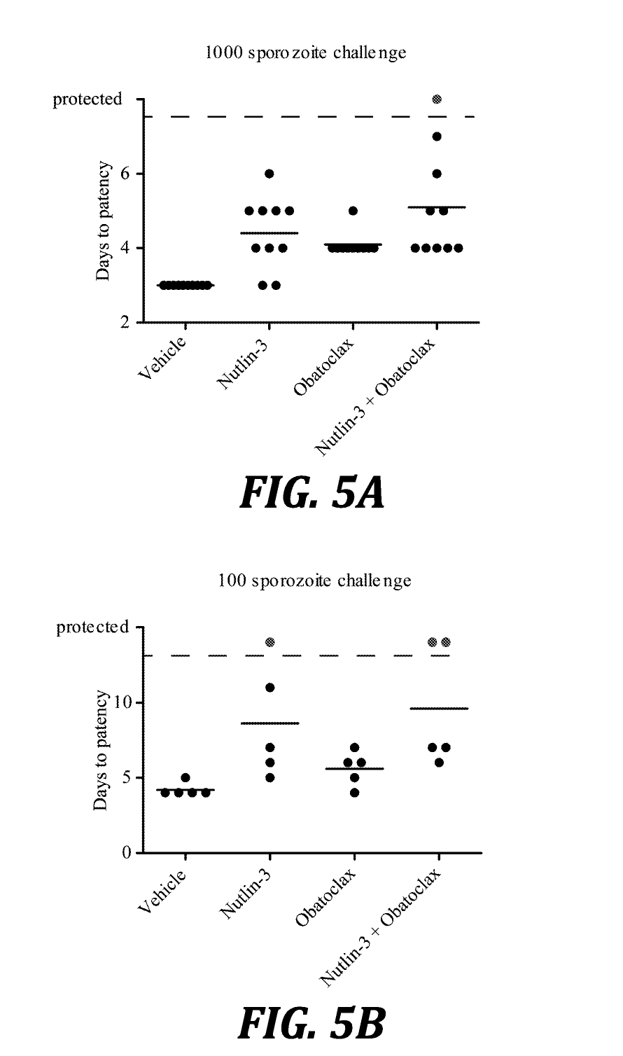

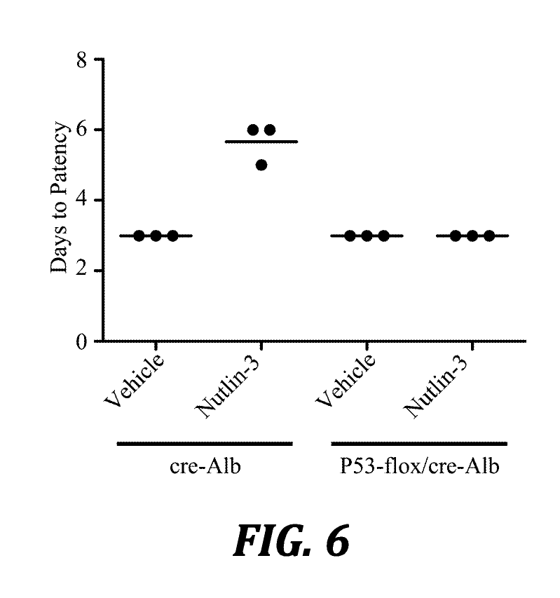

Because a single HBP drug was able to decrease but not completely eliminate parasites, we asked if targeting both the p53 and mitochondrial apoptosis pathways would further curtail LS infection. We chose to assess combination treatments by monitoring the onset of patency in BALB/cJ mice, a highly sensitive way to assess the potency of any drug regimen. BALB/cJ mice were treated with vehicle, Nutlin-3 (twice daily, 200 mg/kg), Obatoclax (once daily, 5 mg/kg), or Nutlin-3 and Obatoclax in combination. Drugs were administered for four days. On the second day of treatment, each mouse was infected with a high (1000 spz) or low (100 spz) P. yoelii challenge by intravenous injection. Mice that received the vehicle became patent on day 3 or day 4, respectively (FIGS. 5A and 5B). Mice that received HBP monotherapy demonstrated a delay in the onset of patency. Patency was delayed even more substantially in mice who received both Nultin-3 and Obatoclax (FIGS. 5A and 5B). In total, three of fifteen mice receiving the combination treatment of Nutlin-3 and Obatoclax did not become patent (FIGS. 5A and 5B). These results indicate that targeting both the Bcl-2 and p53 pathways with HBP drugs can delay, or in some cases completely prevent, the onset of P. yoelii disease in BALB/cJ mice. Considering the demonstration that Nutlin-3 does not clear parasites in an apoptotic-dependent manner (FIGS. 2A-2E), we asked if the impact of Nutlin-3 on Plasmodium liver stage development was entirely dependent on hepatocyte P53. We monitored the onset of patency in both wild-type p53 (cre-Alb) and liver-specific p53 knockout mice (p53-flox/cre-Alb). Mice were treated with vehicle or Nutlin-3 (twice daily, 200 mg/kg by oral gavage) for 4 days. On the second day of treatment, mice were challenged with 1000 spz by intravenous injection. Strikingly, while Nutlin-3 was highly effective in cre-Alb mice, Nutlin-3 did not delay the onset of patency in liver-specific p53 knockout mice (FIG. 6). This demonstrates that hepatocyte p53 is responsible for the elimination of LS parasite after Nutlin-3 treatment.

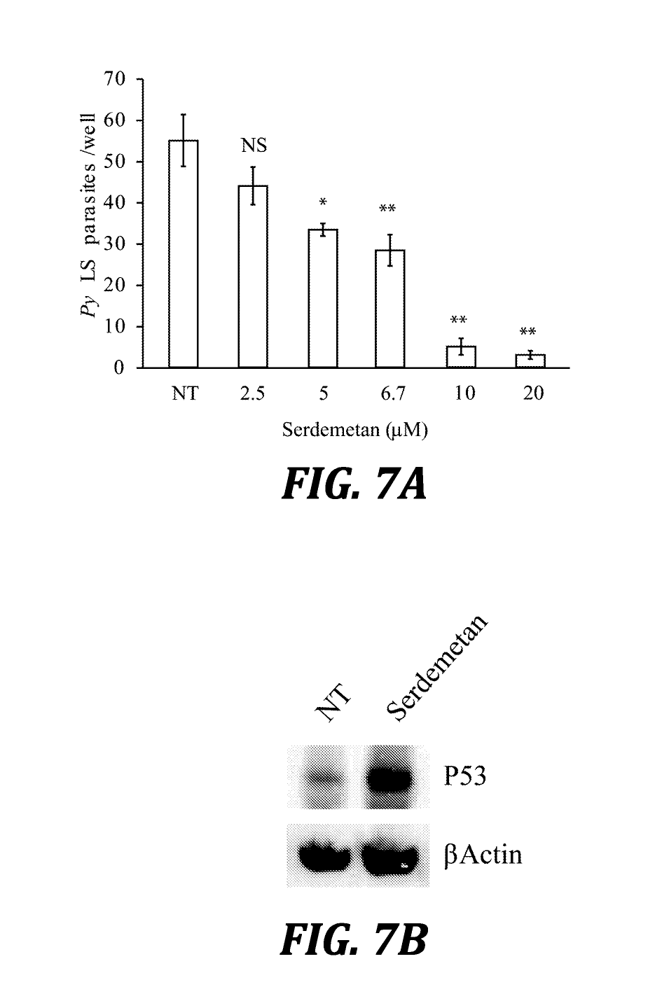

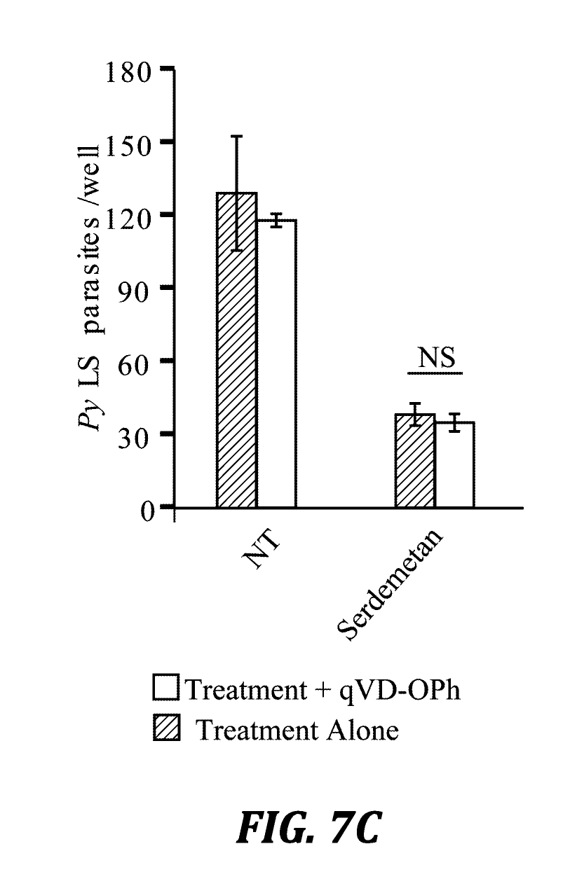

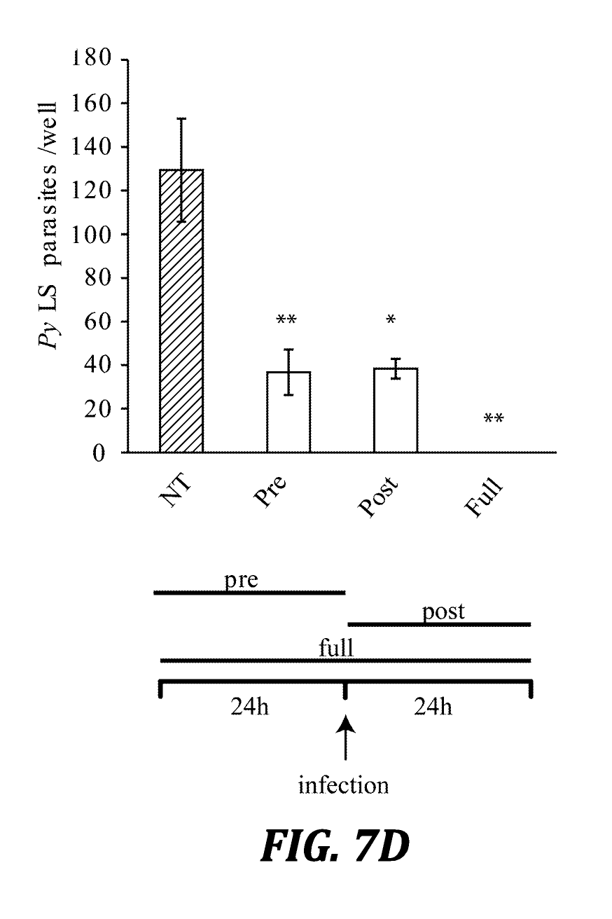

While the combination of Nutlin-3 and Obatoclax treatment substantially impacts the onset of patency, not all mice were protected from developing blood stage infection with this strategy. Thus, we chose to test the efficacy of another p53 agonist, Serdemetan. Serdemetan reportedly has both increased bioavailability and lower toxicity than Nutlin-3 (Yuan, Y., et al., “Novel Targeted Therapeutics: Inhibitors of MDM2, ALK and PARP,” Journal of Hematology & Oncology 4:16 (2011), incorporated herein by reference in its entirety) Serdemetan acts by binding to the RING domain of MDM-2 which minimizes the degradation p53 (Yuan, Y., et al., Journal of Hematology & Oncology 4:16 (2011)) (see FIG. 1B for the chemical structure). Like Nutlin-3, Serdemetan substantially boosted p53 protein levels after 48 hours of treatment (FIG. 7B). In vitro, Serdemetan eliminated more than 90% of LS-infected hepatocytes (P=0.004) with half the dose of Nutlin-3 (FIGS. 2A and 7A). The addition of qVD-OPh did not reverse the effect of Serdemetan, indicating that Serdemetan, like Nutlin-3, eliminates LS-infected hepatocytes in an apoptosis-independent manner (FIGS. 2E and 7C). We have previously shown that increasing p53 levels prior to infection with Nutlin-3 decreased parasite infection rates in vitro (Kaushansky, A., et al., Cell Reports 3:630-637 (2013)). We asked if this was also true with Serdemetan pre-treatment. We treated Hepa 1-6 cells with Serdemetan (10 μM) 24 hours prior to infection with P. yoelii, 24 hours following infection, or 24 hours before and after infection (FIG. 7D). Strikingly, when Hepa 1-6 cells were treated with Serdemetan for 24 hours both before and after infection, LS parasites were completely eliminated (P=0.01).

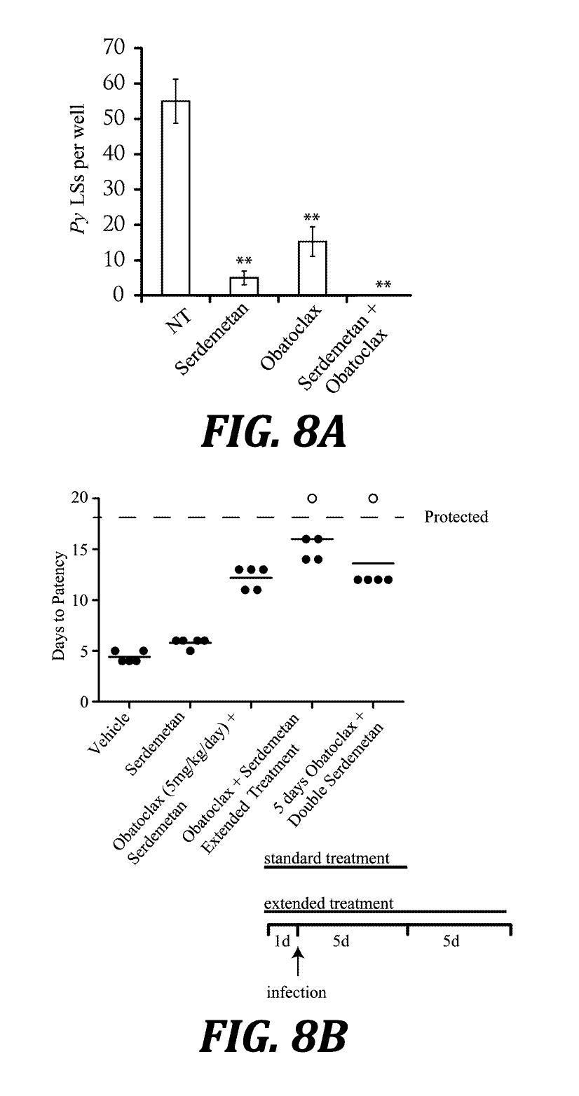

We next treated Hepa 1-6 cells with both Serdemetan and Obatoclax. After 48 hours of treatment, Serdemetan and Obatoclax (P=0.004) in combination completely cleared LS parasites (FIG. 8A). This indicates that activating p53 and inhibiting the Bcl-2 family concurrently is more effective than targeting either pathway alone. To determine if this also held true in vivo, we tested several different conditions, administered once daily beginning 24 hours prior to infection: (1) vehicle only (plus daily for 5 days post-infection), (2) Serdemetan (20 mg/kg/day) (plus daily for 5 days post-infection), (3) Serdemetan (20 mg/kg/day) in combination with Obatoclax (5 mg/kg/day) (plus daily for 5 days post-infection), (4) Serdemetan (40 mg/kg/day) and Obatoclax (5 mg/kg/day) in combination (plus daily for 5 days post-infection), or (5) Serdemetan (20 mg/kg/day) and Obatoclax (5 mg/kg/day) in combination (plus daily for 10 days post-infection) (FIG. 8B). All three of the combination treatment conditions significantly delayed the onset of blood stage patency compared to vehicle or Serdemetan alone (FIG. 8B) or when compared to Obatoclax alone (FIG. 5B). Individuals exhibiting complete protection from the combination treatment groups are indicated with open circles.

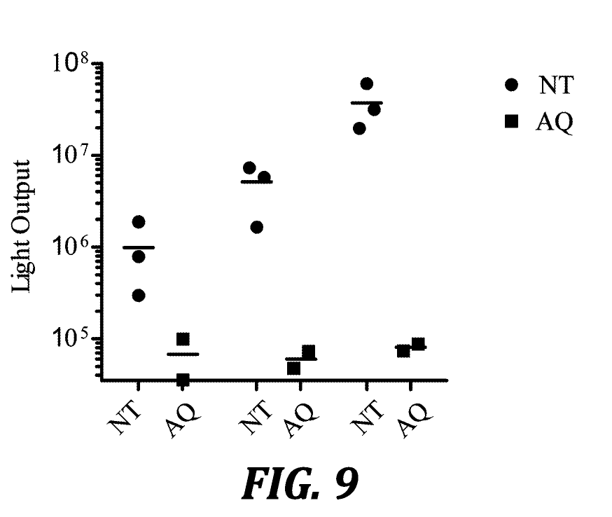

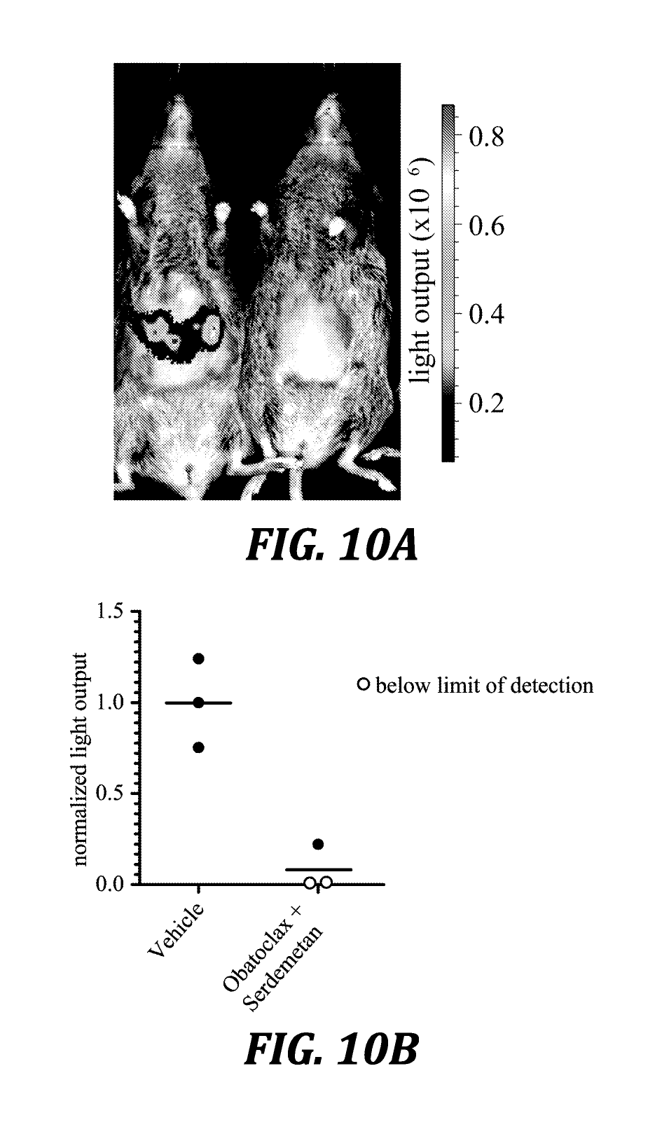

Host-based prophylaxis strategies are based on exploiting critical pathways in the hepatocyte upon which the parasite relies. We and others have demonstrated that the human and rodent infecting Plasmodium parasites do not have entirely overlapping requirements of their host hepatocytes (Kaushansky, A., & Kappe, S. H., “The Crucial Role of Hepatocyte Growth Factor Receptor During Liver-Stage Infection Is Not Conserved Among Plasmodium Species,” Nature Medicine 17:1180-1181 (2011); Silvie, O., et al., “Hepatocyte CD81 is Required for Plasmodium falciparum and Plasmodium yoelii Sporozoite Infectivity,” Nature Medicine 9:93-96 (2003), each incorporated herein by reference in its entirety). This might suggest that host-based prophylaxis strategies directed at human parasites need to be fine-tuned for the specific requirements a parasite species has of its host. Human-infecting P. falciparum parasites spend approximately seven days developing in the liver, as opposed to two days for rodent-infecting species, providing a longer window for liver-targeted prophylaxis. We have recently demonstrated that the FRG HuHep mouse (FAH-/-/Rag2-/-Il2rg-/-), which is T-, B- and NK-cell deficient and is repopulated with >90% human hepatocytes, allows for P. falciparum LS infection (Vaughan, A. M., et al., “Complete Plasmodium falciparum Liver-Stage Development in Liver-Chimeric Mice,” The Journal of Clinical Investigation 122:3618-3628 (2012), incorporated herein by reference in its entirety). However, this model has not yet been used to explore drug efficacy against P. falciparum. As a proof of concept, we used the anti-malarial compound Atovaquone (AQ) to test if the model was useful for drug studies. AQ was administered at 10 mg/kg/day for three days. On the second day of treatment, we infected the animals with 10⁶ P. falciparum sporozoites by intravenous injection and monitored LS burden by luminescence using an in vivo imaging system (IVIS) for 6 days after infection (FIG. 9). Parasite load in mice treated with AQ was kept near the limit of detection for all time points. This confirms previous human studies and indicates that our system is an accurate model by which to test drug regimens. We then extended our humanized mouse work to test the HBP regimen of Serdemetan (20 mg/kg/day) and Obatoclax (5 mg/kg/day). Mice were treated beginning one day before infection and then treated daily for 7 days (FIGS. 10A and 10B). Like AQ, Obatoclax and Serdemetan combination treatment left liver stage burden near the limit of detection (FIGS. 10A and 10B).

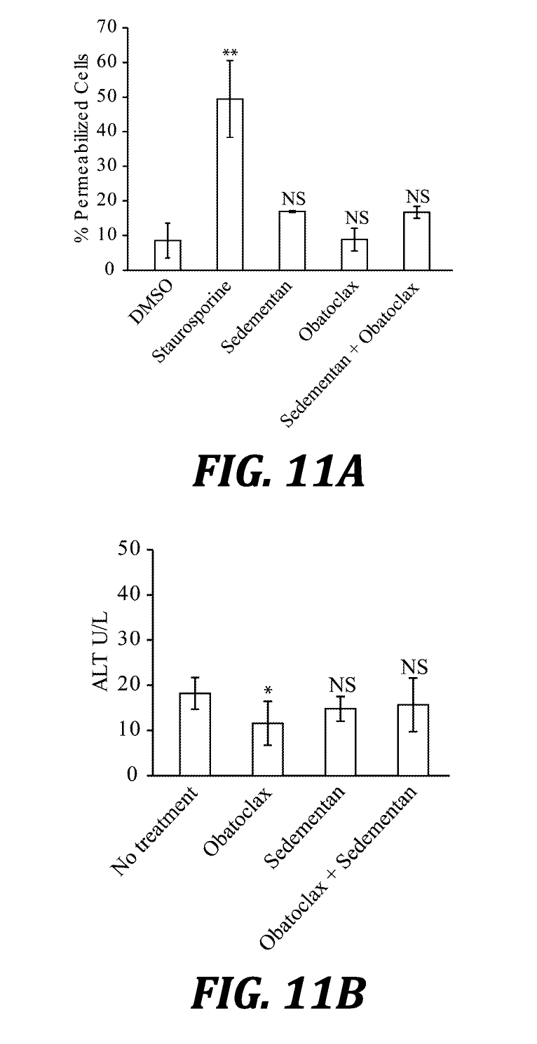

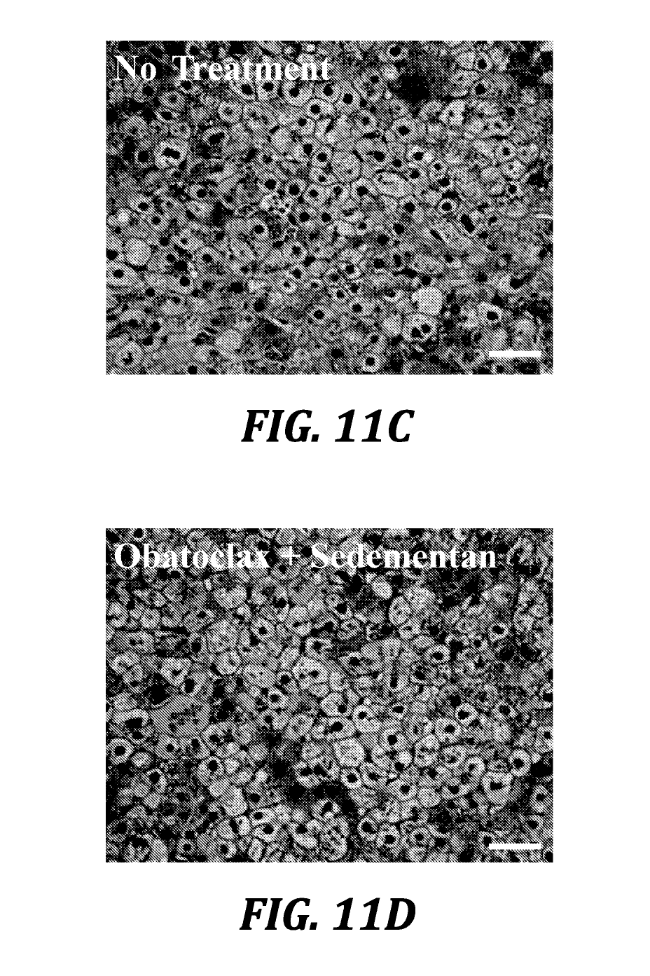

Any prophylactic regimen must also be well-tolerated since it is given to healthy individuals. All HBP drugs described here did not lead to uninfected cell permeabilization in vitro (FIG. 11A), liver damage in BALB/cJ mice (FIG. 11B), or altered liver morphology in FRG HuHep mice (FIGS. 11C and 11D). These data further support the notion that HBP combination treatment does not impact uninfected cells but rather selectively targets Plasmodium-infected hepatocytes. Taken together, these data demonstrate that host based prophylaxis approaches provide a novel approach to prevent the onset of P. falciparum disease.

### DISCUSSION

Plasmodium parasites have evolved strategies to ensure their survival within both the insect vector and the mammalian host (Mackinnon, M. J. & Marsh, K., “The Selection Landscape of Malaria Parasites,” Science 328:866-871 (2010); Crompton, P. D., et al., “Malaria Immunity in Man and Mosquito: Insights Into Unsolved Mysteries of a Deadly Infectious Disease,” Annual Review of Immunology 32:157-187 (2014), each incorporated herein by reference in its entirety). The first critical point for parasite growth in mammals is the liver stage, where a single sporozoite produces tens of thousands of blood-stage infectious parasites within the confines of a single hepatocyte. We have previously shown that the parasite creates an environment for intracellular replication by regulating host hepatocyte levels of the Bcl-2 family of oncogenes and the tumor suppressor p53 (Kaushansky, A., et al., Cell Reports 3:630-637 (2013); Kaushansky, A., et al., Cell Death & Disease 4:e762 (2013)). For decades the accepted model of tumor progression suggested that up-regulation of oncogenes like Bcl-2 and p53 makes cancer cells recalcitrant to treatment. However, recent data suggests that cancers with elevated levels of Bcl-2 oncogenes become ‘addicted’ to this survival pathway and thus are sensitized to its inhibition (Hartman, M. L. & Czyz, M., Anti-cancer Agents in Medicinal Chemistry 12:966-981 (2012); Del Gaizo Moore, V. & Letai, A., “BH3 Profiling-Measuring Integrated Function of the Mitochondrial Apoptotic Pathway to Predict Cell Fate Decisions,” Cancer Letters 332:202-205, (2013), each incorporated herein by reference in its entirety). LS-infected hepatocytes exhibit elevated levels of Bcl-2 and are sensitized to Bcl-2 family inhibition, indicating that the survival of the LS-infected hepatocyte is also characterized by a Bcl-2 family addiction. A comprehensive picture of the specific member(s) of the Bcl-2 family that mediate the protection from apoptosis in LS-infected hepatocytes remains unknown. Our data suggest that Bcl-xL plays a substantial role, since inhibition of Bcl-2 alone with ABT-199 induces minimal decrease in LS burden when compared to inhibiting Bcl-2 and Bcl-xL in combination (FIG. 3). These data are consistent with previous findings that Bcl-xL is a critical mediator of hepatocyte apoptosis (Takehara, T., et al., “Hepatocyte-Specific Disruption of Bcl-xL Leads to Continuous Hepatocyte Apoptosis and Liver Fibrotic Responses,” Gastroenterology 127:1189-1197 (2004), incorporated herein by reference in its entirety).

While inhibition of the Bcl-2 family triggers the intrinsic apoptotic pathway in infected hepatocytes, elevated p53 levels clear infected hepatocytes independently of host apoptosis. p53 suppression can regulate cellular metabolism (Johnson, R. F. & Perkins, N. D., “Nuclear Factor-kappaB, p53, and Mitochondria: Regulation of Cellular Metabolism and the Warburg Effect,” Trends in Biochemical Sciences 37:317-324, (2012); Berkers, C. R., et al., “Metabolic Regulation by p53 Family Members,” Cell Metabolism 18:617-633, (2013), each incorporated herein by reference in its entirety), induce cell cycle progression (Li, T., et al., Cell 149:1269-1283 (2012)) and prevent cellular senescence (Rufini, A., et al., “Senescence and Aging: The Critical Roles of p53,” Oncogene 32:5129-5143 (2013), each incorporated herein by reference in its entirety) in addition to promoting apoptosis. It has been recently demonstrated that p53's functional role in cellular transformation can occur independent of its role in apoptosis, cell cycle arrest and senescence (Li, T., et al., Cell 149:1269-1283 (2012)), further supporting the notion that an alternative p53-driven pathway might be suppressing LS parasite growth and survival.

Our data demonstrate that targeting two independent host pathways is effective at removing both human and rodent infecting LS parasites. Combination treatment with Obatoclax and Serdemetan significantly decreases LS parasite burden and delays the transition to the P. yoelii erythrocytic stage. The effect of the HBP regimen is mirrored in P. falciparum, where Obatoclax and Serdemetan prevent the onset of blood stage parasites in the FRG-HuHep model. To our knowledge, this is the first time the FRG-HuHep model has been used to test a prophylactic regimen for P. falciparum. Moving forward, this model should allow investigators to hone drug regimens prior to clinical trials.

Drugs which target the liver stage may be useful in preventing the onset of P. falciparum disease, yet their impact could be even more substantial for the elimination of P. vivax. Unlike P. falciparum, P. vivax parasites cause relapsing malaria infection by persistence of dormant liver stages (hypnozoites), which resist most treatment regimens (Sibley, C. H., “Understanding drug resistance in malaria parasites: Basic science for public health,” Molecular and Biochemical Parasitology 195:107-114 (2014), incorporated herein by reference in its entirety). The drugs we describe here are designed to exploit parasite requirements of their host cell, rather than the rapidly dividing parasite itself. If hypnozoites have similar requirements of their host cell as replicating liver stages, it is likely that HBP approaches could also eliminate hypnozoites and thus prevent relapsing infection. We have recently determined that the FRG-HuHep model can assess both the presence of replicating and dormant P. vivax liver stages, and now have the necessary tools to determine if HBP strategies are an effective strategy against hypnozoites.

Host-based prophylaxis strategies might prevent the development of drug-resistant parasites. To date, malaria treatments have consisted of compounds that directly target Plasmodium parasites (Sibley, C. H., Molecular and Biochemical Parasitology 195:107-114 (2014)). While resistance to chloroquine took many years to develop, subsequent efforts to design more specifically targeted malaria treatments have also resulted in drug resistance within 5 years of implementation (Wongsrichanalai, C., et al., The Lancet Infectious Diseases 2:209-218 (2002)). Notably, resistance to Atovaquone, a promising drug that eliminates LS parasites, was observed during clinical trials (Wongsrichanalai, C., et al., The Lancet Infectious Diseases 2:209-218 (2002)). The most recent and effective strategy to address parasite resistance are artemisinin combination therapies (ACT), which pairs the quick-acting artemisinin with one of several different partner drugs, such as mefloquine or lumefantrine (Wongsrichanalai, C., et al., The Lancet Infectious Diseases 2:209-218 (2002); Sibley, C. H., Molecular and Biochemical Parasitology 195:107-114 (2014)). Unfortunately, Plasmodium has already developed resistance to artemisinin and its derivatives, as well as each of the partner drugs, when administered on their own (Wongsrichanalai, C., et al., The Lancet Infectious Diseases 2:209-218 (2002); Sibley, C. H., Molecular and Biochemical Parasitology 195:107-114 (2014)). Further complications associated with delivery and administration of malaria treatment, such as drug production cost, fraudulent packaging of medications, and political regulations have also contributed to the swift development of drug resistance to ACTs as they are introduced to the public (Gelband, Hellen, Claire Panosian, and Kenneth J. Arrow, eds. “Saving Lives, Buying Time: Economics of Malaria Drugs in an Age of Resistance,” National Academies Press, (2004); Tabernero, P., et al., “Mind the Gaps—the Epidemiology of Poor-Quality Anti-Malarials in the Malarious World—Analysis of the WorldWide Antimalarial Resistance Network Database,” Malaria Journal 13:139 (2014), each incorporated herein by reference in its entireties). All of these factors point to an urgent need for radically different methods to prevent further drug resistance. Because the drugs we describe here target the non-dividing hepatocyte, we hypothesize such an approach would substantially decrease the capacity of the parasite to mutate to become drug resistant. Furthermore, clinical trials have demonstrated that Obatoclax and Serdemetan, as well as next-generation p53 agonists and Bcl-2 inhibitors, are well-tolerated in humans (Tabernero, J., et al., “A Phase I First-in-Human Pharmacokinetic and Pharmacodynamic Study of Serdemetan in Patients With Advanced Solid Tumors,” Clinical Cancer Research: An Official Journal of the American Association for Cancer Research 17:6313-6321 (2011); Chiappori, A. A., et al., “A Phase I Trial of pan-Bcl-2 Antagonist Obatoclax Administered as a 3-h or a 24-h Infusion in Combination With Carboplatin and Etoposide in Patients With Extensive-Stage Small Cell Lung Cancer,” British Journal of Cancer 106:839-845 (2012); Oki, Y., et al., “Experience With Obatoclax Mesylate (GX15-070), a Small Molecule pan-Bcl-2 Family Antagonist in Patients With Relapsed or Refractory Classical Hodgkin Lymphoma,” Blood 119:2171-2172 (2012), each incorporated herein by reference in its entirety). Thus, transition to the clinic of HBP might come with fewer roadblocks than de novo drug discovery efforts.

Host-based therapies to combat infectious disease are not unique to the malaria liver stage. Targeting host signaling pathways in order to eliminate or prevent infection has been investigated for various pathogens, including M. tuberculosis (Fauci, A. S. & Challberg, M. D., “Host-Based Antipoxvirus Therapeutic Strategies: Turning the Tables,” The Journal of Clinical Investigation 115:231-233 (2005); Hawn, T. R., et al., “Host-Directed Therapeutics for Tuberculosis: Can We Harness the Host?” Microbiology and Molecular Biology Reviews: MMBR 77:608-627 (2013); Law, G. L., et al., “Systems Virology: Host-Directed Approaches to Viral Pathogenesis and Drug Targeting,” Nature Reviews: Microbiology 11:455-466 (2013); Lee, S. M. & Yen, H. L., “Targeting the Host or the Virus: Current and Novel Concepts for Antiviral Approaches Against Influenza Virus Infection,” Antiviral Research 96:391-404 (2012); Yang, H., et al., “Antiviral Chemotherapy Facilitates Control of Poxvirus Infections Through Inhibition of Cellular Signal Transduction,” The Journal of Clinical Investigation 115:379-387 (2005), each incorporated herein by reference in its entirety), which, like P. vivax, is challenging to eliminate due to slowly dividing and dormant forms. Anti-viral regimens targeting host signaling pathways are under development for the treatment of severe influenza infection (Lee, S. M. & Yen, H. L., Antiviral Research 96:391-404 (2012)). Obatoclax has been shown to clear Influenza A and B viral infections at similar doses to those used in this study (Denisova, O. V., et al., “Obatoclax, Saliphenylhalamide, and Gemcitabine Inhibit Influenza A Virus Infection,” The Journal of Biological Chemistry 287:35324-35332 (2012), incorporated herein by reference in its entirety). The comprehensive understanding of host responses to divergent pathogens might provide a universal set of factors upon which several pathogen species rely. This information could be exploited to develop HBP strategies which target multiple pathogens with a single drug regimen, and revolutionize drug treatment in resource-sparse settings which face the challenge of a multitude of co-infecting and deadly infectious diseases.

Materials and Methods

Cell lines and culture. Hepa 1-6 Cells were obtained from ATCC. Cells were maintained in Dulbecco's Modified Eagle Medium (DMEM) complete media (Cellgro, Manassas, Va., USA), supplemented with 10% FBS (Sigma-Aldrich, St. Louis, Mo., USA), 100 IU/ml penicillin (Cellgro), 100 mg/ml streptomycin (Cellgro), and 2.5 mg/ml fungizone (HyClone/Thermo Fisher, Waltham, Mass., USA), and split 1-2 times weekly. Where indicated, cells were treated with ABT-737 (Selleck Chemicals, Houston, Tex., USA), Obatoclax mesylate (Selleck Chemicals), Nutlin-3 (Selleck Chemicals), Serdemetan (JNJ-26854165, Selleck Chemicals) and/or the pan-caspase inhibitor Q-VD-OPh (N-(2-Quinolyl)-L-valyl-L-aspartyl-(2,6-difluorophenoxy) methylketone, SM Biochemicals LLC, Anaheim, Calif., USA) at indicated concentrations. All molecules were dissolved in DMSO for cell culture experiments. Final concentration of DMSO did not exceed 0.5%.

Mosquito rearing and sporozoite production. For P. yoelii sporozoite production, female 6-8 week old Swiss Webster mice (Harlan, Indianapolis, Ind., USA) were injected with blood stage P. yoelii (17XNL) parasites to begin the growth cycle. Animal handling was conducted according to the Institutional Animal Care and Use Committee-approved protocols. We used infected mice to feed female Anopheles stephensi mosquitoes after gametocyte exflagellation was observed. We isolated salivary gland sporozoites according to the standard procedures at days 14 or 15 post blood meal. For each experiment, salivary glands were isolated in parallel in order to ensure that sporozoites were extracted from salivary glands under the same conditions.

For P. falciparum sporozoite production, in vitro P. falciparum NF54HT GFP-luc blood-stage cultures were maintained in RPMI 1640 (25 mM HEPES, 2 mM 1-glutamine) supplemented with 50 μM hypoxanthine and 10% A+ human serum in an atmosphere of 5% CO2, 5% 02, and 90% N2. Cells were subcultured into O+ erythrocytes. Gametocyte cultures were initiated at 5% hematocrit and 0.8%-1% parasitemia (mixed stages) and maintained for up to 17 days with daily medium changes. Non-blood fed adult female Anopheles stephensi mosquitoes 3-7 days after emergence were fed on gametocyte cultures. Gametocyte cultures were quickly centrifuged at 300×g, and the pelleted infected erythrocytes diluted to a 40% hematocrit with fresh A+ human serum and O+ erythrocytes. Mosquitoes were allowed to feed through Parafilm for up to 20 minutes. Following blood feeding, mosquitoes were maintained for up to 19 days at 27° C., 75% humidity, and provided with 8% dextrose solution in PABA water. Infection prevalence was checked at days 7-10 by examining dissected midguts under light microscopy for the presence of oocysts with salivary gland dissections performed at days 14-19.

Quantification of LS parasites after ABT-737, ABT-199, Obatoclax, Nutlin-3, or Serdemetan treatment by manual counting. In all, 1.5×10⁵ Hepa 1-6 cells were seeded in DMEM complete medium in each well of an eight-well Permanox slide. Cells were infected with 5×10⁴ P. yoelii sporozoites. Slides were centrifuged for 3 min at 515× g in a hanging-bucket centrifuge to aid in sporozoite invasion. After 90 min, we removed media that contained sporozoites that had not infected the cells and added fresh media only, or media containing ABT-737 (100 nM), ABT-199 (100 nM), Obatoclax (100 nM), Nutlin-3 (20 μM), or Serdemetan (10 μM) with or without qVD-OPh (20 μM). We allowed LSs to develop for 24 hours or 48 hours, at which time cells were fixed with 4% paraformaldehyde, blocked, and permeabilized for 1 hour in PBS with the addition of 0.1% Triton X-100 and 2% BSA. Staining steps were performed in PBS supplemented with 2% BSA. We stained cells using anti-sera to Plasmodium HSP70 and UIS4 proteins at 4° C. overnight and then washed several times, and antibodies were visualized with the use of AlexaFluor-488 goat anti-mouse and AlexaFluor-594 goat anti-rabbit secondary antibody (Life Technologies, Grand Island, N.Y., USA). We used DAPI stain to visualize both hepatocyte and parasite nuclei. Sporozoites that had not invaded and/or developed in hepatoma cells were distinguished by UIS4 circumferential staining and morphology. All LS parasites in each well were counted, and each assay was performed in biological triplicate.

Nutlin-3 and Obatoclax in vivo experiments. 60 BALB/cJ mice (Jackson Laboratory, Bar Harbor, Me., USA) were treated with either vehicle control 5 mg/kg of Obatoclax, 200 mg/kg Nutlin-3, or both Obatoclax and Nutlin-3 by oral gavage once (Obatoclax) or twice (Nutlin-3) daily for 3 days. On the second day of treatment, mice were injected with 1000 or 100 P. yoelii sporozoites. Patency was checked by Giemsa-stained thin blood smear daily for the first 7 days, then every other day until day 14. Animal handling was conducted according to the Institutional Animal Care and Use Committee-approved protocols.

Specific Action of Nutlin-3 in vivo experiment. Liver-specific p53 knock-out mice were generated by breeding p53 floxed mice (B6.129P2-Trp53tm1Brn/J) and cre-albumin (B6.Cg-Tg(Alb-cre)21Mgn/J). P53 floxed/unfloxed alleles were identified by size of band on a 2% agarose gel (270 bp for WT, 390 bp for floxed) using the primers: forward—GGT TAA ACC CAG CTT GAC CA (SEQ ID NO:1) and reverse—GGA GGC AGA GAC AGT TGG AG (SEQ ID NO:2). Cre was identified by the presence/absence of the mutant allele (heterozygotes vs. homozygotes were not distinguished) using the primers: forward—GAA GCA GAA GCT TAG GAA GAT GG (SEQ ID NO:3) and reverse—TTG GCC CCT TAC CAT AAC TG (SEQ ID NO:4). All DNA was extracted using Qiagen DNEasy Blood & Tissue Kit (Qiagen Inc., Valencia, Calif., USA) per manufacturer's protocol. PCR was performed using BioMix Red (2×) from BioLine (BioLine USA Inc., Taunton, Mass., USA).

Both wild-type p53 (cre-alb) and liver-specific p53 knockout mice (cre-alb/flox-p53) were treated with either vehicle or Nutlin-3 as described in the previous in vivo method. On the second day of treatment, all mice were infected i.v. with 1000 P. yoelii sporozoites. Patency was checked by Giemsa-stained thin blood smear daily until all mice became patent. Animal handling was conducted according to the Institutional Animal Care and Use Committee-approved protocols.

Serdemetan and Obatoclax in vivo experiments with P. yoelii infection. BALB/cJ mice (Jackson Laboratory, Bar Harbor, Me., USA) were treated with either vehicle control 5 mg/kg of Obatoclax, 20 mg/kg Serdemetan, or both Obatoclax and Serdemetan by oral gavage once daily for 5 days. On the second day of treatment, mice were injected with 10⁵ P. yoelii sporozoites. Patency was checked by giemsa-stained thin blood smear daily for the first 7 days, then every other day until day 14. Animal handling was conducted according to the Institutional Animal Care and Use Committee-approved protocols.

In vivo experiments with P. falciparum infection in humanized mice. Atovaquone study: FRG HuHep mice were treated with either vehicle or Atovaquone (10 mg/mL, Sigma-Aldrich) by oral gavage once daily for three days. On the second day of treatment, mice were infected (i.v.) with 10⁶ P. falciparum GFP-luc transgenic parasites. To quantify infection, mice were injected with 100 μl of XenoLight RediJect-d-Luciferin (PerkinElmer), anesthetized and then imaged within 5 minutes of injection using the IVIS® Lumina II animal imager (Perkin Elmer) with a 10 cm field of view, medium binning factor and an exposure time of up to 5 min. Infection was monitored by IVIS on days 4, 5, and 6 post-infection.

HBT study: FRG HuHep mice were treated with either vehicle control or both Obatoclax (5 mg/kg) and Serdemetan (20 mg/kg) by oral gavage once daily for 8 days. On the second day of treatment, mice were injected with 10⁶ Plasmodium falciparum GFP-luc transgenic parasites. On day 7 (6 days post-infection), liver stage burden was assessed by IVIS as described above.

Quantification of p53 by Western Blotting. We plated 10⁶ Hepa 1-6 cells per well of a 6-well plate in DMEM complete media and treated with complete media only, 20 μM Nutlin-3, 100 nM ABT-737, 100 nM Obatoclax, or 10 μM Serdemetan for 24 or 48 hours. Cells were lysed in SDS lysis buffer (2% SDS, 50 mM Tris-HCl, 5% glycerol, 5 mM EDTA, 1 mM NaF, 10 mM β-glycerophosphate, 1 mM PMSF, 1 mM activated Na₃VO₄, 1 mM DTT, 1% phosphatase inhibitor cocktail 2; Sigma-Aldrich), 1% PhosSTOP Phosphatase Inhibitor Cocktail Tablet (Roche), filtered overnight at 3000 rpm through AcroPrep Advance Filter Plates (Pall Corporation) and stored at −80° C. Western blots were performed according to manufacturer instruction with the iBlot Dry Transfer System (Life Technologies, Carlsbad, Calif., USA) using an antibody to p53 (Clone 1C12; Cell Signaling Technology) and then normalized to signal from an anti-β-actin (Cell Signaling Technology) Western blot. Signals from immunoblots were detected using either an Alexa 680-conjugated anti-rabbit antibody or an Alexa 800-conjugated anti-mouse antibody (LI-COR Biosciences). Membranes were visualized using an Odyssey infrared imaging system (LI-COR Biosciences).

ALT Assay. Mice were treated with vehicle, 5 mg/kg Obatoclax, 20 mg/kg Serdemetan, or both Obatoclax and Serdemetan for 5 days by oral gavage, and 200 μL blood was taken from each mouse 2 weeks after drug administration was completed. The blood was allowed to clot at room temperature for 30 minutes then centrifuged for 5 minutes at 3,300× g. The cleared sera were then analyzed for ALT levels with the Alanine Aminotransferase (ALT/SGPT)-SL kit (Sekisui Diagnostics, Charlottetown, PE, Canada) according to manufacturer instructions. Briefly, R1 and R2 were combined at 4:1 and warmed to 37° C. before addition to a clear, flat-bottom 96-well plate already containing sera samples. The plate was allowed to incubate for 5 minutes at 37° C. before NADH consumption was analyzed for 10 minutes at 340 nm with a SpectraMax M2 plate reader (Molecular Devices, LLC, Sunnyvale, Calif.) using SoftMax Pro software. ALT U/L was then determined using equation 1.

$$ALT = \frac{Δ A \text{/} min \times Assay Volume ( mL ) \times 1000}{6.22 \times Light Patch ( cm ) \times Sample Volume ( mL )} . \qquad Equation 1$$

Cell Permeabilization Assay. Hepa 1-6 cells were seeded in a 24-well tissue culture-treated plate at 3×10⁵ cells per well overnight. Cells were treated for 48 hours with DMSO (0.1%), Obatoclax (100 nM), Serdemetan (10 μM), or Obatoclax and Serdemetan and then analyzed for cell death using the LIVE/DEAD® Fixable Yellow Dead Cell Stain Kit (Molecular Probes, Life Technologies, Grand Island, N.Y., USA) as per manufacturer instructions. Briefly, cells were stained live with reconstituted dye (1:500) in 500 μL PBS on ice for 30 minutes, mixing periodically, washed twice with 500 μL PBS, and then fixed with 100 μL BD Cytofix/Cytoperm™ fixation/permeabilization (BD Biosciences, San Jose, Calif., USA) solution for 15 minutes on ice. Cells were stored in 200 μL 5 mM EDTA in PBS at 4° C. until analyzed using a BD™ LSR II flow cytometer (BD Biosciences) at 605 nm on the violet 405 nm laser, and resulting FCS files were analyzed with FlowJo 7.6.1 (Tree Star, Ashland, Oreg., USA).

Statistical Analysis. Where appropriate, statistical significance was determined using a two-tailed, unpaired t test, assuming unequal variance. A p-value ≦ 0.05 is indicated by *, a p-value ≦ 0.01 is indicated by **, and non-significant p-values (≧ 0.05) are indicated by NS. Unless otherwise noted, each group was compared to the DMSO or non-treated control samples.

The following is a description of the surprising discovery that the combination of Bcl2-inhibitors and p53 agonists results in eradication of hypnozoite stages of Plasmodium vivax from host liver cells in vivo. These results demonstrate that a combined therapy of Bcl2-inhibitors and p53 agonists is useful for host-based clearance of P. vivax and prevention of the relapse observed for Plasmodium species that produce hypnozoites.

Rationale

As indicated above, certain species of Plasmodium, such as P. malariae, P. ovale and P. vivax can produce distinct liver stage parasites, called hypnozoites, which lie dormant or latent within the liver cells. The hypnozoites are able to reactivate after a period of time and proceed along the life-cycle progression, thus leading to clinical symptoms of the disease even after a significant passage of time since the initial infection via the mosquito vector. Furthermore, the hypnozoites are notoriously resistant to most treatment regimens that are used to clear active Plasmodium stages. The persistence of any hypnozoites throughout treatment can lead to relapse of the disease in a subject, even if the subject initially appeared to have been cleared of the infection. Thus, the recalcitrance of hypnozoite stages to standard malaria therapies remains a major obstacle to the long-term treatment of the disease and well-being of the infected subject.

Results

As described above, we demonstrated that a host-based prophylactic (HBP) approach that combined administration of Bcl2-inhibitors and p53 activators dramatically reduce, and even eliminate, liver stage burden both in vitro and in vivo. While drugs that successfully target the liver stage parasites by affecting the host-cells may be useful in preventing the onset of P. falciparum disease, their impact on long-term disease treatment could be even more substantial for the elimination of P. vivax and other Plasmodium infections that are capable of hypnozoite-driven relapse. As described above, the HBP therapeutic approach strategy exploits parasites' requirements of their host cell, rather than attack the rapidly dividing parasite itself. If hypnozoites have similar requirements of their host cell as replicating liver stages, it is likely that the described herein could also negatively affect the viability of hypnozoite stages residing in the host liver cells.

As described above, we have recently shown that the FRG-HuHep model can assess both the presence of replicating and dormant P. vivax liver stages. To determine if the host-based prophylaxis approaches are effective against P. vivax, we treated FRG HuHep mice with Obatoclax and Serdemetan and infected the mice with P. vivax, according to the general protocols described above. Briefly, three FRG HuHep mice were treated with either vehicle or 5 mg/kg of Obatoclax and 20 mg/kg Serdemetan by oral gavage once daily for 10 days. After the first 24 hours of the initial treatment, we infected the mice with 10⁶ P. vivax sporozoites. At eight days post-infection, the mice were sacrificed and liver cells were examined by microscopic examination for the presence and quantification of schizont and hypnozoite LS stages. With this technique, hypnozoites are readily distinguishable from schizonts by virtue of their comparative sizes.

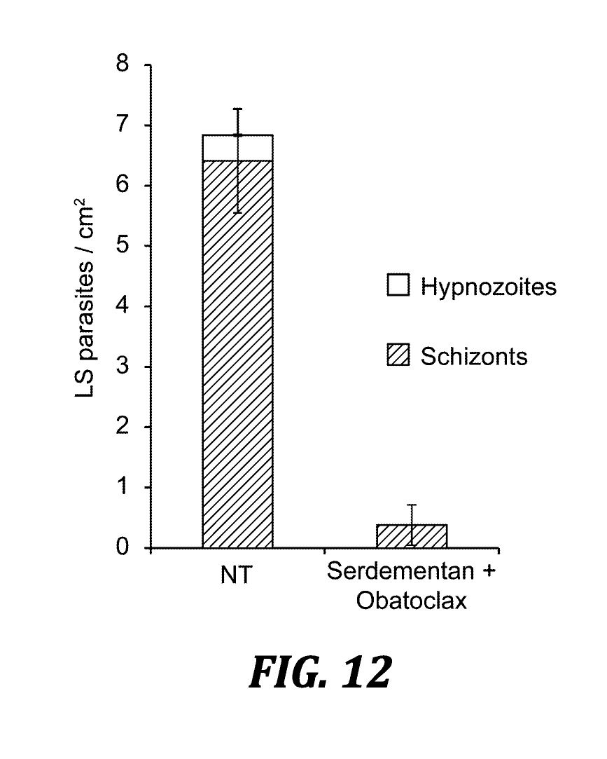

As illustrated in FIG. 12, administration of Obatoclax and Serdemetan significantly reduced the burden of schizont LSs. Surprisingly, the impact is even more impressive against P. vivax hypnozoites. FIG. 12 illustrates that the administration of combined administration of Obatoclax and Serdemetan results in the complete elimination of hypnozoite stages from the liver cells. This result is unprecedented and has profound implications for the long-term treatment of these Plasmodium parasites because relapse can only be prevented with the complete clearance of hypozoites stages. Thus, the present data demonstrates the utility of the described HBP treatment strategy for the complete elimination of heretofore recalcitrant cases of relapsing malaria infections.

While illustrative embodiments have been illustrated and described, it will be appreciated that various changes can be made therein without departing from the spirit and scope of the invention.

1. A method for inhibiting growth or development of a liver-stage Plasmodium parasite in a hepatocyte, comprising administering to the hepatocyte an effective amount of at least one pro-apoptotic agent and an effective amount of at least one p53 activator.

2. The method of claim 1, wherein the at least one pro-apoptotic agent promotes the mitochondrial apoptotic cascade.

3. The method of claim 2, wherein the at least one pro-apoptotic agent inhibits expression or function of a Bcl-2 family protein.

4. The method of claim 3, wherein the at least one pro-apoptotic agent inhibits functional binding of the BH-3 domain of the Bcl-2 family protein.

5. The method of claim 3, wherein the Bcl-2 family protein is Bcl-xL or a homolog thereof.

6. The method of claim 3, wherein at least one pro-apoptotic agent inhibits the expression of the Bcl-2 family protein and is a histone deacetylase inhibitor, a retinoid, a cyclin-dependent kinase inhibitor, or any analog thereof, or an antisense nucleic acid molecule targeting a gene encoding the Bcl-2 family protein.

7. The method of claim 3, wherein at least one pro-apoptotic agent is gossypol, ABT-737, ABT-263, an indole bipyrrole such as GX15-070, HA14-1, antimycin, obatoclax, isoxazolidine, benzoyl urea, AT-101, TW-37, or any functional derivative or analog thereof.

8. The method of claim 1, wherein the at least one p53 activator increases the stability, expression, or activity of p53.

9. The method of claim 8, wherein the at least one p53 activator is or includes 9AA, a canbinol, an HLI98 series molecule, a JJ78:1/12 series molecule, a tenovin, CDB3, KCG165, an aminothiosol, or RITA, as recited in Table 1.

10. The method of claim 1, wherein the at least one p53 activator inhibits or reduces the interaction of p53 with Mdm2 or MdmX.

11. The method of claim 10, wherein the at least one p53 activator is or includes a benzodiazepine, a benzodiazepinedone, a chromenotrizolopyrimindine, a dehydroaltenusin, an imidazole-indole, a spiro-oxindole, an imidazoline, an oxindole, a spiroindolinone, an isoquinolines, a bisaryl sulfonamide, a substituted piperidine, a diphenyl-dihydro-imidazopyridinone, an imidazothiazole, a deazaflavin, an isoindolin-1-one, boronic acid, a pyrrolidin-2-one, SJ172550, or a tryptamine, as recited in Table 1.

12. The method of claim 11, wherein the at least one p53 activator is or includes Nutlin-3 or Serdemetan.

13. The method of claim 1, wherein the effective amount of the at least one pro-apoptotic agent and/or the effective amount of the at least one p53 activator is administered prior to exposure of the hepatocyte to a Plasmodium parasite.

14. The method of claim 1, wherein the effective amount of the at least one pro-apoptotic agent and/or the effective amount of the at least one p53 activator is administered concurrently with or subsequent to exposure of the hepatocyte to a Plasmodium parasite.

15. The method of claim 1, wherein the effective amount of the at least one pro-apoptotic agent is administered concurrently with the effective amount of the at least one p53 activator.

16. The method of claim 1, wherein the therapeutically effective amount of the at least one pro-apoptotic agent is administered prior to or subsequent to the administration of the effective amount of the at least one p53 activator.

17. The method of claim 1, wherein the liver-stage Plasmodium parasite is P. falciparum, P. vivax, P. ovale, P. malariae, P. knowlesi, P. yoelii, P. berghei, P. chabaudi, P. vinckei, or P. cynomolgi.

18. The method of claim 17, wherein the liver-stage Plasmodium parasite is a hypnozoite of P. vivax or P. ovale.

19. The method of claim 1, wherein the liver-stage Plasmodium parasite is a drug-resistant Plasmodium parasite.

20. The method of claim 1, wherein the hepatocyte is cultured in vitro and the effective amounts of the at least one pro-apoptotic agent and the at least one p53 activator are administered to the culture.

21. The method of claim 1, wherein the hepatocyte is in vivo in a vertebrate subject and the effective amounts of the at least one pro-apoptotic agent and the at least one p53 activator are administered to the vertebrate subject.

22. The method of claim 21, wherein the vertebrate is infected with a Plasmodium parasite or is susceptible to infection with a Plasmodium parasite.

23. The method of claim 21, wherein the vertebrate subject is a human subject.

24. The method of claim 1, wherein inhibiting growth or development of a liver-stage Plasmodium parasite results in the elimination of the liver-stage Plasmodium parasite from the hepatocyte.

25. A method of inhibiting growth or development of a liver-stage Plasmodium parasite in a hepatocyte of a vertebrate subject, comprising administering to the subject an effective amount of at least one pro-apoptotic agent and an effective amount of at least one p53 activator.

26. A method of preventing infection of a hepatocyte in a vertebrate subject by a liver-stage Plasmodium parasite, comprising administering to the subject an effective amount of at least one pro-apoptotic agent and an effective amount of at least one p53 activator.

27. A method of preventing or reducing production of blood-stage Plasmodium parasite by a liver-stage Plasmodium parasite in a hepatocyte of a vertebrate subject, comprising administering to the subject an effective amount of at least one pro-apoptotic agent and an effective amount of at least one p53 activator.

28. A method of generating protective immunity against a Plasmodium parasite in a vertebrate subject, comprising administering to the subject an effective amount of at least one pro-apoptotic agent and an effective amount of at least one p53 activator.

29. The method of any one of claims 25-28, wherein the effective amounts of the at least one pro-apoptotic agent and/or the at least one p53 activator are administered to the subject prior to, concurrently with, or subsequent to infection of a hepatocyte of the subject with the Plasmodium parasite.

30. The method of any one of claims 25-28, wherein the vertebrate subject is a human.

31. The method of claim 25 or claim 27, wherein the liver-stage Plasmodium parasite is a hypnozoite of P. vivax or P. ovale.

32. The method of claim 31, wherein the administration of the effective amounts of the at least one pro-apoptotic agent and the at least one p53 activator results in elimination of the hypnozoites from the subject.
